(19) United States
(12) Patent Application Publication
(10) Pub. No.: US 2025/0084366 A1
(43) Pub. Date: Mar. 13, 2025

(54) SUPPLEMENTED SERUM-FREE MEDIA INCLUDING UNHYDROLYZED PLANT PROTEIN FOR CULTURED MEAT PRODUCTION

(71) Applicant: Trustees of Tufts College, Medford, MA (US)

(72) Inventors: David L. Kaplan, Medford, MA (US); Andrew Stout, Medford, MA (US)

(21) Appl. No.: 18/954,053

(22) Filed: Nov. 20, 2024

Related U.S. Application Data
(63) Continuation of application No. PCT/US2023/067320, filed on May 22, 2023.
(63) Continuation of application No. PCT/US2023/067321, filed on May 22, 2023.
(60) Provisional application No. 63/344,522, filed on May 20, 2022.
(60) Provisional application No. 63/344,522, filed on May 20, 2022.

(51) Int. Cl. C12N 5/00

(52) U.S. Cl. CPC C12N 5/0031; C12N 2500/05; C12N 2500/30; C12N 2500/46; C12N 2501/105; C12N 2501/12; C12N 2501/135; C12N 2501/2306; C12N 2501/998; C12N 2501/999

(57) ABSTRACT

A serum-free culture media for expansion of the number of cells and related method of use are disclosed. The disclosed media can be for use in cultured food applications. The disclosed media can include a baseline serum-free and animal component-free culture media and a plant protein composition. The plant protein composition can comprise unhydrolyzed plant proteins. The plant protein composition can comprise plant proteins that are 3 kDa in size or greater. The plant protein composition can be present in the serum-free cell culture media at a final concentration of 0.05 g/L to 1 g/L. The baseline media, in use, provides a baseline growth capability for expansion of the numbers of muscle satellite cells for use in cultured food applications. The disclosed media, in use, provides an improved growth capability for expansion of the number of muscle satellite cells. Methods of making and using the disclosed media are also disclosed.

### CROSS-REFERENCE TO RELATED APPLICATIONS

This application is a continuation of application nos. PCT/US2023/067320, filed May 22, 2023, and PCT/US2023/067321, filed May 22, 2023, both of which claim priority to U.S. Provisional Application No. 63/344,522 filed on May 20, 2022. The contents of each of which are incorporated by reference in their entirety.

### STATEMENT REGARDING FEDERALLY FUNDED RESEARCH

This invention was made with government support under P41EB002520 awarded by National Institutes of Health and 2021-69012-35978 by the U.S. Department of Agriculture. The government has certain rights in the invention.

### BACKGROUND

Cell-cultured meat is an emerging technology which offers both promising possibilities and significant scientific challenges. The promise of cultured meat lies in its potential to address environmental, ethical, and human health issues that plague intensive animal agriculture. For instance, limited life-cycle analyses suggest that cultured meat could require >90% less land and >75% less water than conventional beef, while contributing >75% fewer greenhouse gas emissions, >95% less cutrophication, and >90% less particulate matter formation. At the same time, cultured meat could improve animal welfare, food-system resilience, and human health outcomes. The challenges that face the successful technological transition of cultured meat to the marketplace stem from the need for production systems that are low-cost, scalable, food-safe, and free of animal-derived inputs. Here, cell culture media is a particularly problematic hurdle for several reasons. First, media comprises the majority (>99%) of the cost of current production systems. Second, the culture of meat-relevant cells, such as bovine satellite cells (BSCs), has traditionally relied on fetal bovine serum (FBS), a notoriously expensive, unsustainable, and inconsistent component, which is inherently antithetical to the aims of cultured meat. Finally, when scrum-free media for satellite cells have been explored, they are either complex, ineffective compared to serum-containing media, reliant on proprietary or animal-derived additives, contain components (e.g., synthetic steroids) that could raise regulatory concerns, or contain overly-expensive components which are incompatible with economic cultured meat production. As such, despite the substantial body of work that has gone into the exploration of satellite cell culture systems, a low-cost, food-safe, and fully animal-derived component-free medium capable of sustained expansion of satellite cells remains a crucial limitation for the field.

Recently, the inventors and colleagues addressed some of these challenges by developing a new culture media, termed “Beefy-9”, which includes a recombinant form of albumin, as described in International Patent Application No. PCT/US2021/062668, which is incorporated herein in its entirety by reference. While that culture media provided good results, the recombinant form of albumin itself is quite expensive, so the composition remains at least somewhat cost prohibitive.

As a result, a need exists for new culture media that are capable of achieving performance that rivals existing culture media, but with the use of ingredients that are significantly less expensive than recombinant albumin.

### SUMMARY

In some aspects, the present disclosure provides a serum-free cell culture media for expansion of cells, the media comprising a basal culture media, a plant protein composition comprising a majority of plant proteins that are 3 kDa or greater in size at a final concentration of 0.05 g/L to 1 g/L in the serum-free cell culture media. The serum-free cell culture media is free or substantially free of albumin. The plant protein composition in the serum-free cell culture media was not treated with a hydrolytic enzyme or other hydrolytic process, and/or at least about 70% of the plant proteins are 10 kDa or greater in size.

In some aspects, the plant protein composition is from a plant selected from the group consisting of oilseed crops, non-oilseed oil crops, legumes, pulses, and combinations thereof, and in some aspects, the plant protein composition is preferably from and/or includes an oilseed crop. The plant protein composition may be from rapeseed, soybean, sunflower seed, sesame seed, flax seed, camelina, safflower, linseed, grapeseed, pumpkin seed, poppyseed, watermelon seed, and combinations thereof. In some aspects, the plant protein composition comprises protein prepared from rapeseed. The plant protein composition may also include proteins from a non-oilseed oil crop. The plant protein composition may include proteins from palm kernel, coconut, olive, corn, hemp, almond, cashew, pea, chickpea, fava bean, lentil, lupin, lima bean, mung bean, navy bean, Bambara bean, mesquite bean, mucuna bean, pigeon pea, potato bean, yam bean, and combinations thereof. In some aspects, the plant protein composition is present in the serum-free cell culture media at a concentration of at least 50 mg/L, at least 100 mg/L, at least 200 mg/L, at least 250 mg/L, at least 500 mg/L, at least 750 mg/L, at least 1 g/L, and at most 50 g/L, at most 25 g/L, at most 15 g/L, at most 10 g/L, at most 5 g/L, at most 2 g/L, at most 1.5 g/L, or at most 1 g/L. In some aspects, the plant protein composition is present in the serum-free cell culture media at a preferable concentration of about 200 mg/L to about 400 mg/L.

In some aspects, the serum-free cell culture media comprises a basal culture media. Basal media may contain ingredients essential for cell survival and growth including amino acids, sugar(s) such as glucose, vitamins, minerals, buffers and ions (calcium, magnesium, potassium, sodium, and phosphate, for instance). The serum-free cell culture media may be the B8 media used and defined in the examples. In some aspects, the serum-free cell culture media comprises a basal culture media that is selected from the group comprising Dulbecco's Modified Eagle Medium (DMEM), Ham's F12 media, Roswell Park Memorial Institute (RPMI) 1640 media, Leibovitz's L15 and a mixture thereof. The media may further comprise recombinant proteins important for sustaining the growth of the cells or other additives to support growth of the cells. The serum-free cell culture media may contain no animal-derived components. The serum-free cell culture media may include recombinantly derived animal proteins. In some aspects, the serum-free cell culture media is food-grade.

In some aspects, the present disclosure provides methods of making a serum-free cell culture media. The method comprises adding a plant protein composition at a final concentration of 0.05 g/L to 1 g/L in the serum-free cell culture media to a basal culture media to produce the serum-free cell culture media. The plant protein composition and the basal culture media used in the method may be any of those described here. The plant protein composition used in the serum-free cell culture media may be prepared using methods of plant protein preparation. Briefly, a ground plant starting material, which may be a defatted or partially defatted plant material may be solubulized in an aqueous solution comprising water.

In some aspects, the present disclosure provides methods of expanding the numbers of or differentiating cells. The method comprises adding cells to the serum-free cell culture media disclosed herein, incubating the cells in the serum-free cell culture media to allow expansion of the number of or differentiation of cells and production of growth media, and harvesting the cells and/or growth media. The method may further comprise, prior to adding the cells, either: i) coating a cell culture substrate or the cells with a cell adhesive peptide; or ii) adhering the cells to the cell culture substrate in the basal culture media and/or a different basal culture media that is lacking the plant protein composition. The method may further comprise both the coating of step i) and the adhering of step ii). The cells may be genetically engineered cells. In some aspects, the cells expand more when incubated in the serum-free cell culture media than control cells incubated in the basal culture media without the plant protein composition. The cells may expand at least 50% more when incubated in the serum-free cell culture media than control cells incubated in the basal culture media without the plant protein composition. The cells may expand in the serum-free cell culture media provided herein comparably to cells grown in media comprising recombinant albumin. The methods may further comprise using the harvested cells in a cultured food product. The method may further comprise using the growth media to produce a food or pharmaceutical product. The cells for use in the methods are selected from muscle satellite cells, fibroblasts, adipogenic precursor cells, mesenchymal stem cells, embryonic stem cells, induced pluripotent stem cells or combinations thereof. The cells preferably include muscle satellite cells. The muscle satellite cells may be primary cells or cells that have been immortalized through genetic modification or spontaneous immortalization. The muscle satellite cells may be bovine, galline, ovine, porcine, equine, murine, caprine, lapine, or piscine. In the examples, the cells are bovine muscle satellite cells.

### BRIEF DESCRIPTION OF THE DRAWINGS

The patent or application file contains at least one drawing executed in color. Copies of this patent or patent application publication with color drawing(s) will be provided by the Office upon request and payment of the necessary fec.

FIG. 1. Protein isolate generation. (A) The methods used for generating oilseed protein isolates (OPIs) involved alkali extraction, isoelectric precipitation, centrifugation, and filtration in order to generate concentrated protein solutions (50 mg/mL). The resulting OPIs were clear or reddish-brown in color, and slightly viscous. (B) Comparisons of global annual protein meal supply and cost, unoptimized protein yields, and OPI cost based on yields and starting protein meal cost (excluding processing costs from inputs such as chemicals, energy costs, filters, etc.). The results showed substantially reduced cost for soybean protein isolate (SPI), rapeseed protein isolate (RPI) and cottonseed protein isolate (CPI) compared to Inca peanut protein isolate (IPPI). (C) SDS-PAGE of OPIs next to corresponding ladder for each protein. The results revealed heterogeneous mixtures of proteins <75 kDa, with larger average protein fractions for SPI and CPI compared with IPPI and RPI.

FIG. 2. Short-term growth in OPI-supplemented serum-free media. (A) Four-day cell growth in B8 medium supplemented with various OPIs compared with B8 supplemented with 0.8 mg/mL recombinant albumin (Beefy-9). The results showed that SPI, RPI, and CPI significantly improved cell growth compared to no supplementation, but only RPI was able to recover cell growth comparable to Beefy-9 (1.15 times the growth of Beefy-9, no significant difference). n=6 distinct samples; statistical significance was calculated by multiple unpaired t-tests between control (“0”) and OPI concentration with the most growth, as well as between OPI concentration with the most growth and Beefy-9. A Bonferroni correction was applied (α=0.025) to account for multiple comparisons. Statistical significance is indicated for p<0.01 (**), p<0.001 (***), p<0.0001 (****). (B) Brightfield images of bovine satellite cells (BSCs) on day three of growth in Beef-R or Beefy-9 showed no difference in cell morphology between conditions. Scale bars are 200 μm.

FIG. 3. Multi-passage growth in Beefy-R. (A) Four-passage cell growth in various media. The results showed that Beefy-R improved growth over Beefy-9, though not to the degree of BSC-GM. n=3 distinct samples; statistical significance was calculated by two-way ANOVA and is indicated for the final timepoint with p<0.01 (**). (B) Doubling time calculations for cell growth in (A). While growth slowed for all media types over the duration of the experiment, results showed that Beefy-R maintained doubling times <24 h for two passages, compared with one for Beefy-9 and three for BSC-GM. n=3 distinct samples; statistical significance was calculated by two-way ANOVA and is indicated for p<0.05 (*), p<0.01 (**).

FIG. 4. Phenotypic analysis of cells cultured in various media. (A) qPCR of proliferative cells (Pro.) cultured in various media, and in cells following two days of differentiation (Diff.). In proliferating cells, results showed increased MyoD expression for BSC-GM and increased Myogenin expression for Beefy-R and Beefy-9. In differentiated cells, results showed increased MyoD, Myogenin, and MHC expression in Beefy-R and Beefy-9 compared with BSC-GM. n=3 distinct samples; statistical significance was calculated by two-way ANOVA (or one-way ANOVA for Myogenin Pro. insert) and is indicated for p<0.05 (*), p<0.01 (**), p<0.001 (***), p<0.0001 (****). (B) Immunostaining of proliferative cells in Beefy-R for nuclei (DAPI, blue), Pax7 (magenta), and MyoD (green). The results showed ubiquitous staining for Pax7 and heterogeneous staining for MyoD. Similar staining for Beefy-9 and BSC-GM is given in FIG. 9. Scale bars are 200 μm. (C) Pax7 quantification revealed >99% Pax7+ for all three media types (Beefy-R=99.98%; Beefy-9=99.70%; BSC-GM=99.34%). n=12 (4 images each for 3 distinct samples). (D) Immunostaining of differentiated cells from Beefy-R for nuclei (DAPI), myosin heavy chain (MHC) (magenta), and actin (green). Results showed robust formation of MHC-positive multinucleated myotubes. Similar staining for Beefy-9 and BSC-GM is given in FIG. 11. Scale bars are 200 μm. (E) Fusion index analysis of differentiated myotubes revealed significantly enhanced differentiation for Beefy-R and Beefy-9 compared with BSC-GM. n=13-15 images for one distinct sample; statistical significance was calculated by two-way ANOVA and is indicated for p<0.01 (**).

FIG. 5. Growth with immortalized BSCs in Beefy-R prepared with multiple lots of rapeseed protein cakes. (A) Short-term growth screen. Both lots of RPI showed improved growth compared with B8 (0 mg/mL of RPI supplementation). Lot #2 showed optimal growth at 0.2 mg/mL, and both lots showed a significant improvement over B8 at this concentration, though Lot #2 showed significantly reduced growth compared with Lot #1. n=3 distinct samples; statistical significance was calculated by multiple unpaired t-tests between RPIs at 0.2 mg/mL, as well between RPIs of each lot at 0.2 mg/mL and B8 (0 mg/mL) controls. A Bonferroni correction was applied (α=0.025) to account for multiple comparisons. Statistical significance is indicated for p<0.025 (*) and p<0.001 (***). (B) Month-long immortalized BSC growth in serum-free media. The results showed that Beefy-R produced with 0.2 mg/mL of RPI from Lot #1 of rapeseed protein cakes (sourced in 2021) demonstrated improved growth compared with Beefy-R produced with Lot #2 rapeseed cakes (sourced in 2022). All serum-free media showed continuous growth over one month for iBSCs. n=3 distinct samples.

FIG. 6. Gene ontology (GO) classification of proteins in OPIs, weighted by Normalized Spectral Abundance Factor (NSAF). (A) GO terms of proteins classified under “Biological Process”. The results showed increased prevalence of “biological regulation,” “cellular process,” and “metabolic process” proteins in SPI and RPI compared with CPI, and increased concentrations of “developmental process,” “multicellular organismal process,” “reproduction,” and “reproductive process” proteins in CPI. (B) GO terms of proteins classified under “Cellular Component”. The results showed increased prevalence of “cytoplasm” proteins for SPI and RPI compared with CPI, increased concentrations of “endoplasmic reticulum” proteins for SPI, and increased concentrations of “membrane” proteins for CPI. (C) GO terms of proteins classified under “Molecular Function”. The results showed increased concentrations of “binding” and “catalytic activity” proteins for SPI and RPI compared with CPI, and increased concentrations of “nutrient reservoir activity” proteins for CPI.

FIG. 7: Comparison of RPI extraction methods. (A) RPI extracted using various methods (original described in Materials and Methods, original method plus an overnight incubation at 4° C. after the filtration step, original method plus an overnight incubation at 4° C. and with 120.6 mM NaCl added to the protein solution after the filtration step, original method plus an initial hexane defatting step for one hour and an overnight incubation at 4° C. before the extraction protocol. Results showed no clear difference in cell growth of trends for the different extraction methods. (B) Comparing the optimum concentrations of the four extraction methods mentioned previously (0.4 or 0.2 mg/mL) revealed no significant difference in cell growth between any methods. n=6 distinct samples; statistical significance was calculated by one-way ANOVA and is indicated for p >0.2 (n.s.).

FIG. 8: Short-term growth of BSCs with other potential albumin alternatives. (A) Cyclodextrins showed no improvement over cell growth compared with B8, and significantly reduced growth compared with Beefy-9. Analysis performed via Presto Blue metabolic assay on days 3 and 4 of growth. n=3 distinct samples; statistical significance was calculated by one-way ANOVA for day 4 samples compared with Beefy-9 and is indicated for p<0.0001 (****). (B) Supplier-provided plant protein hydrolysates showed no improvement over cell growth compared with B8, and significantly reduced growth compared with Beefy-9. Analysis performed via Presto Blue metabolic assay on days 3 and 4 of growth. n=3 distinct samples; statistical significance was calculated by one-way ANOVA for day 4 samples compared with Beefy-9 and is indicated for p <0.0001 (****). (C) Dextran 500 showed no improvement B8. PEG 10k showed minimal improvement at high concentrations. Both conditions showed significantly reduced growth compared with albumin-containing Beefy-9. Analysis performed via FluoReporter dsDNA quantification after days 3 and 4 of growth. n=6 distinct samples; statistical significance was calculated by one-way ANOVA for day 4 samples compared with Beefy-9 and is indicated for p <0.0001 (****).

FIG. 9: Immunostaining of proliferative BSCs (p3) in various media. Staining for nuclei (DAPI, blue), Pax7 (magenta), and MyoD (green). The results showed ubiquitous staining for Pax7 and heterogeneous staining for MyoD for all media types. Qualitative comparison showed a higher degree of bright MyoD staining for BSC-GM cells compared with Beefy-R and Beefy-9, which supports qPCR data seen in FIG. 4. Scale bars are 200 μm.

FIG. 10: Example Pax7 quantitation. Example image represents one of 12 that was used for calculating Pax7-positive percentage in all media types. Top left: DAPI (nuclear) staining. Bottom left: DAPI staining converted to binary (black and white) and used to define nuclear regions. Top right: Pax7 staining. Bottom right: Pax7 staining with threshold applied and DAPI regions applied (yellow circles).

FIG. 11: Immunostaining of differentiated BSCs (p3) in various media. Cells were differentiated for two days. Staining for nuclei (DAPI, blue), Myosin Heavy Chain (MHC; magenta), and Actin (green). Results showed robust myotube formation for all media types. Scale bars are 200 μm.

FIG. 12: Example fusion analysis. Example image represents one of 12 that was used for calculating fusion index in all media types. Top left: MHC staining. Bottom left: MHC staining with threshold applied and used to define region of interest (yellow outline). Top right: DAPI (nuclear) staining. Bottom right: DAPI staining with region of interest applied. This image was used to calculate the ratio of nuclei within yellow-defined myotube regions compared with total nuclei.

FIG. 13: Lipid accumulation in BSCs cultured in various media. BSCs (P5) from various media conditions were imaged via brightfield microscopy to observe lipid droplet accumulation, which appears as bright circles within cells. Results showed that while some lipid droplet formation still occurred in Beefy-R (examples indicated by black arrows; left image), it is substantially reduced compared with Beefy-9, which showed substantial lipid accumulation and the formation of large clusters of lipid droplets in cells (examples indicated by black arrows & black circles; middle image). BSC-GM controls showed very little to no aberrant lipid accumulation in cells. Scale bars are 100 μm.

FIG. 14: Short-term growth of Mack1 (A) and LS adapted Mack1 (B) in rapeseed protein isolate (RPI)-supplemented serum-free media. RPI supplementation can replace FBS in Mack1 and LS adapted Mack 1 cell cultures as measured by dsDNA quantification via fluorescence. Statistical significance was determined by multiple comparisons test between 0 (control) and RPI with the most growth (P<0.05, *; P<0.001, ***; and P<0.0001, ****).

### DETAILED DESCRIPTION

Before the present invention is described in further detail, it is to be understood that the invention is not limited to the particular embodiments described. It is also understood that the terminology used herein is for the purpose of describing particular embodiments only and is not intended to be limiting. The scope of the present invention will be limited only by the claims. As used herein, the singular forms “a”, “an”, and “the” include plural embodiments unless the context clearly dictates otherwise.

It should be apparent to those skilled in the art that many additional modifications beside those already described are possible without departing from the inventive concepts. In interpreting this disclosure, all terms should be interpreted in the broadest possible manner consistent with the context. Variations of the term “comprising” should be interpreted as referring to elements, components, or steps in a non-exclusive manner, so the referenced elements, components, or steps may be combined with other elements, components, or steps that are not expressly referenced. Embodiments referenced as “comprising” certain elements are also contemplated as “consisting essentially of” and “consisting of” those elements. When two or more ranges for a particular value are recited, this disclosure contemplates all combinations of the upper and lower bounds of those ranges that are not explicitly recited. For example, recitation of a value of between 1 and 10 or between 2 and 9 also contemplates a value of between 1 and 9 or between 2 and 10. Further, as used herein, ranges that are between two particular values should be understood to expressly include those two particular values. For example, “between 0 and 1” means “from 0 to 1” and expressly includes 0 and 1 and anything falling inside these values. Also, as used herein “about” means ±20% of the stated value, and includes more specifically values of ±10%, ±5%, ±2%, ±1%, and ±0.5% of the stated value.

In one aspect, the present disclosure provides a serum-free and animal-component-free culture media. The culture media provided here is based off the discovery that a recently-developed serum-free and animal-product-free culture media for induced pluripotent stem cells, which may have been suitable for commercial-scale growth and expansion of muscle satellite cells but includes at least one cost-prohibitive ingredient (in this case, recombinant albumin), could be modified by the substitution of a plant protein extract, concentrate or isolate (i.e., composition) for a recombinant form of albumin for growth and expansion of muscle satellite cells. As used herein, “expansion” and “expand” refers to increasing the number of cells via replication. In other words, by replacing the recombinant form of albumin from Beefy-9 (and potentially adjusting concentrations, as appropriate) with plant proteins, a less expensive and at least as effective (under at least some conditions) culture media was deployed. The culture media may be suitable for commercial production of cultured muscle cells suitable for use in food applications, including human food applications. As used herein, the term “serum-free” refers to media that does not contain animal sera (for example, fetal bovine serum, sheep serum, horse serum). More generally, the terms “animal-free”, “animal-component-free”, and “animal-product-free” refer to compositions, methods, and/or uses which do not introduce animal-derived components (animal serum or animal albumin, for non-limiting examples). In some cases recombinantly derived animal proteins may be employed. In some cases, the recombinantly derived animal proteins are free or substantially free of albumin or free or substantially free of all serum derived proteins). In some cases, the culture media or the baseline culture media on which it is based is free or substantially free of albumin. As used herein, “albumin” refers to animal-derived albumin unless expressly described otherwise (e.g., “plant albumin” or “2S albumin”).

Basal media may contain ingredients essential for cell survival and growth including amino acids, sugar(s) such as glucose, vitamins, minerals, buffers and ions (calcium, magnesium, potassium, sodium, and phosphate. for instance). Suitable baseline or basal culture media is commonly known in the art, non-exclusive examples of which include B8, DMEM, F12, RPMI, L15 and mixtures thereof. Any commercially available basal cell-culture media may be used in the methods and those skilled in the art will appreciate that certain media may be preferable for certain cell types. As used herein, a culture media is “substantially free” of, for instance, albumin, if the removal of whatever small portion of albumin may be present in the culture media reduces performance of the culture media by less than 5%. In some cases a culture media is “substantially free” of, for instance, albumin, if albumin is present in a weight concentration of equal to or less than 1%, 0.5%, 0.1%, 0.05% or 0.01%. In some cases a culture media is “substantially free” of albumin if albumin is present in an amount equal to or less than 0.1 g/L, or 0.01 g/L. The same would apply to being substantially free of serum derived proteins.

The plant protein composition can be present in an amount of at least 50 mg/L, at least 100 mg/L, at least 200 mg/L, at least 250 mg/L, at least 500 mg/L, at least 750 mg/L, at least 1 g/L, at least 1.5 g/L, at least 2 g/L, at least 5 g/L, at least 10 g/L, or at least 15 g/L and at most 50 g/L, at most 25 g/L, at most 15 g/L, at most 10 g/L, at most 5 g/L, at most 2 g/L, at most 1.5 g/L, or at most 1 g/L. In some cases, the plant protein composition is present in an amount of about 1000 mg/L, which in prior work resulted in optimal growth performance over 4 day cell cultures. In some cases, the plant protein composition is present in an amount of between about 100 mg/L and 2000 mg/L. The plant protein composition may preferably be present at a concentration of about 200 mg/L to about 400 mg/L.

The plant protein composition can be extracted, concentrated, or isolated (i.e., collected) from oilseed crops, other oil crops (i.e., non-oilseed oil crops), legumes or pulses, and combinations thereof. As used herein, the material from which the plant protein composition is extracted, concentrated, or isolated from can be raw plant material, such as seeds or the like; processed plant material, such as meals or cakes or the like; plant protein powders or the like; or defatted plant materials or the like; or the like, as would be understood by a skilled artisan.

In some cases, the plant protein composition is extracted, concentrated, or isolated from an oilseed crop. Examples of suitable oilseed crops include, but are not limited to, rapeseed, soybean, sunflower seed, sesame seed, flax seed, camelina, safflower, linseed, grapeseed, pumpkin seed, poppyseed, watermelon seed, and the like. In some specific cases, the oilseed crop can be rapeseed. In some cases, the plant protein composition is extracted, concentrated, or isolated from another oil crop (i.e., an oil-generating crop that is not an oilseed crop). Examples of suitable other oil crops include, but are not limited to, peanut, palm kernel, coconut, olive, corn, hemp, almond, cashew, and the like. In some cases, the plant protein composition is extracted, concentrated, or isolated from legumes or pulses. Examples of suitable legumes or pulses include, but are not limed to, pea, chickpea, fava bean, lentil, lupin, lima bean, mung bean, navy bean, Bambara bean, mesquite bean, mucuna bean, pigeon pea, potato bean, yam bean, and the like.

In some cases, the plant protein composition comprises primarily or mostly unhydrolyzed plant proteins. As used herein, the term “unhydrolyzed” refers to proteins that have not been subjected to hydrolysis via chemical (e.g., enzymatic, acidic or alkaline), heat, mechanical, etc.) or other means. Hydrolysis refers to the process by which peptide bonds are broken under certain conditions (for example, acidic conditions) at certain temperatures, generating shorter protein fragments, peptides, and/or free amino acid residues. Hydrolysis often results in smaller peptides (via the cleavage of one larger peptide or protein). Therefore, one way to recognize unhydrolyzed proteins is by size. For example, a skilled artisan could set a size threshold, above which are larger proteins said to be “unhydrolyzed”. For example, proteins that are 3 kDa or greater in size. For example, proteins that are 7 kDa or greater in size. For example, proteins that are 10 kDa or greater in size. A skilled artisan will recognize that some natural hydrolysis may occur in proteins or during the plant protein preparation process. Additionally, induced hydrolysis can occur with the treatment of a hydrolytic enzyme. Non-exclusive examples of hydrolytic enzymes include proteinase, neutral proteinase, metalloproteinase, those hydrolytic enzymes extracted from Bacillus subtilis, trypsin, those extracted from porcine pancreas, and more.

Hydrolysis can also be identified by the lack of proteins present in a protein composition. In other words, unhydrolyzed protein compositions can be identified by the presence of certain proteins. For example, a plant protein composition that comprises unhydrolyzed plant proteins may comprise plant albumin (2S albumin or other seed storage proteins) and plant globulins (7S globulin, 11S globulins, or other globulin proteins). In some cases, 2S albumins make up 10-40% of plant protein compositions. In some cases, 7S globulins make up 10-20% of plant protein compositions. In some cases, 11S globulins make up 10-40% of plant protein compositions. Oil body-associated proteins and olcosins may also contribute to protein abundance in plant protein compositions. In some cases, 2S albumins, 7S globulins, 11S globulins, oil body-associated proteins, and/or oleosins make up the first-, second-, third-, or fourth-most abundant proteins in plant protein compositions. In some cases, the plant protein composition comprises a majority of plant proteins that are 3 kDa or greater in size, which may or may not have been treated with a hydrolytic enzyme. The plant protein composition may comprise plant proteins wherein at least about 50%, 60%, 70% 74%, 75%, 80%, 85%, 90%, or 95% of the plant proteins are 4, 5, 6, 7, 8, 9, or 10 kDa or greater in size. In some cases, the plant protein composition may comprise plant proteins wherein at least about 70%, 72%, 74%, 75%, or more of the plant proteins are 10 kDa or greater in size.

In some cases, the serum-free and animal-component-free culture media can further include one or more of the following: interleukin 6; ethanolamine; curcumin; oleic acid; minerals; pigment epithelium-derived factor (PEDF); epidermal growth factor (EGF); leukemia inhibitory factor (LIF); hepatocyte growth factor (HGF); insulin-like growth factor 1 (IGF-1); platelet-derived growth factor BB (PDGF-BB); and linoleic acid. The serum-free and animal-component-free culture media may further include one or more of the following: a recombinant version of interleukin 6; ethanolamine; curcumin; oleic acid; or linoleic acid.

The serum-free and animal-component-free culture media and the baseline or basal culture media can include basal media in an amount by weight of at least 80% and at most 99.99%. In some cases, the basal media may be present in an amount by weight of at least 60%, 70%, 80%, or 90%.

The baseline serum-free and animal-component-free culture media can contain a mixture of basal media (e.g., DMEM, DMEM/F12, etc.) containing sugars (e.g., glucose) at concentrations ranging from about 0.01-10 g/L, inclusive; amino acids (e.g., glutamine, lysine) at concentrations ranging from about 0.001-5 g/L, inclusive; vitamins (e.g., folate, niacin) at concentrations ranging from about 0.001-1 g/L, inclusive; minerals (e.g., NaCl) at concentrations ranging from about 0-15 g/L, inclusive; and trace elements (e.g., iron, selenium) at concentrations ranging from about 0.0001-10 mg/L, inclusive. The basal media can be supplemented with growth-stimulating or cell-signaling factors (e.g., insulin, fibroblast growth factor, transforming growth factor, etc.) at concentrations ranging from about 0-100,000 ng/mL, inclusive, and by carrying or transport proteins (e.g., transferrin) at concentrations ranging from about 0-1 g/L, inclusive. In some cases, the culture media can include growth-stimulating or cell-signaling factors and/or carrying or transport proteins at lower concentrations than would ordinarily be present in the baseline culture media. For example, without wishing to be bound by any particular theory, it is believed that the culture media can include growth-stimulating or cell-signaling factors and/or carrying or transport proteins at lower concentrations than would ordinarily be present in the baseline culture media. The inventors surprisingly discovered that, with the inventive culture media, the concentration of growth-stimulating or cell-signaling factors and/or carrying or transport proteins in the culture media may be capable of being zero. In some cases, growth-stimulating factors are excluded from the media. In some cases, cell-signaling factors are excluded from the media. In some cases, carrying proteins are excluded from the media. In some cases, transport proteins are excluded from the media. Additionally, the concentration of minerals in the culture media may be zero.

The serum-free and animal-component-free culture media has a baseline growth capability for expansion of cells for use in cultured food applications. However, this baseline growth capability is unsuitable for commercial growth of cultured food. The disclosed serum-free and animal-component-free culture media has an improved growth capacity that is greater than the baseline growth capacity. The term “baseline growth capacity” may refer to growth in media lacking animal-derived or recombinant albumin. The growth capability may be equivalent to or improved in comparison to growth of the same cells or cell type in media containing animal-derived or recombinant albumin. For example, improved growth capacity that is greater than the baseline growth capacity may be growth that is comparable to or improved as compared to growth in media containing animal-derived or recombinant albumin. In some cases, the improved growth capacity is at least 50%, at least 100%, at least 150%, at least 200%, at least 250%, or at least 300% greater than the baseline growth capacity. In some cases the improved growth capacity is comparable to or about the same as when cells are grown in similar basal media with albumin in place of plant protein. The media provided herein are capable of growing cells for use as a food source for animals including for use as a food source for humans. The cells may be harvested after expansion in the media provided herein for use in food applications. Alternatively or in addition, products made by the cells and released into the growth media may also be collected, harvested, optionally isolated and used in food or pharmaceutical applications.

The baseline and improved growth capacity can be expressed in a variety of ways. As one example, the baseline and improved growth capacity can be expressed as a short-term growth capacity, measured as proliferation over the course of a short period of time, such as 1, 2, 3, 4, 5, 6, or 7 days. As another example, the baseline and improved growth capacity can be expressed as a long-term growth capacity, measured as a number of cell doublings over multiple passages of muscle satellite cells. As a further example, the baseline and improved growth capacity can be expressed as a biomass increase over a given period of time. As yet another example, the baseline and improved growth capacity can be expressed as a percentage of actively doubling cells in culture at a given time (e.g., through cell-cycle analysis). The specific way in which the growth capacity is expressed is not intended to be limiting. Improved growth capacity may also be an indication of the physiological health of the expanded cell population. As demonstrated in the Examples, the cells grown in the media provided herein do not show aberrant lipid accumulation and lipid droplet formation as do cells grown in recombinant albumin. Thus, improved cellular physiology is also a means of improved growth capacity as used herein.

In certain aspects, the culture media can include FGF-2 at a lower concentration than would ordinarily be present in the baseline culture media. In the baseline culture media, the FGF-2 concentration is typically around 40 ng/mL. The inventors surprisingly discovered that, with the inventive culture media, the concentration of FGF-2 in the culture media can be less than 20 ng/mL, less than 15 ng/ml, less than 10 ng/ml, less than 7.5 ng/ml, less than 5 ng/ml, or less than 2.5 ng/mL to obtain similar growth and cell physiology.

In certain aspects, the culture media can include transforming growth factor (TGFβ3) at a lower concentration than would ordinarily be present in the baseline culture media to obtain similar growth functionality. In some cases, the culture media can include TGFβ3 at a concentration of less than 0.1 ng/ml, less than 0.01 ng/mL, less than 0.001 ng/mL. In some cases, the culture media can include no TGFβ3.

In certain cases, the culture media can include neuregulin (NRG1) at a lower concentration than would ordinarily be present in the baseline culture media to obtain similar growth functionality. In some cases, the culture media can include NRG1 at a concentration of less than 0.1 ng/mL, less than 0.01 ng/mL, less than 0.001 ng/mL. In some cases, the culture media can include no NRG1.

In some cases, the culture media may comprise 2-Phospho-L-ascorbic acid trisodium salt, insulin, Transferrin, sodium selenite, fibroblast growth factor 2, Neregulin 1, transforming growth factor beta 3 and mixtures thereof, and, in some cases, insulin, fibroblast growth factor 2, Neregulin 1, and transforming growth factor beta 3 are present at concentrations less than or equal to 10 μg/mL, 20 ng/mL, 0.1 ng/mL, and 0.1 ng/mL, respectively.

In some cases, the culture media can include other components, including recombinant components, at a lower concentration than would ordinarily be present in the baseline culture media to obtain similar growth functionality. For example, without wishing to be bound by any particular theory, it is believed that the culture media can include insulin at a lower concentration than would ordinarily be present in the baseline culture media. In the baseline culture media, the insulin concentration is typically around 20 μg/mL. The inventors surprisingly discovered that, with the inventive culture media, the concentration of insulin in the culture media may be capable of being less than 10 μg/mL, less than 8 μg/mL, less than 7.5 μg/mL, less than 6 μg/mL, less than 5 μg/mL, less than 3 μg/mL, or less than 2 μg/mL. The inventors surprisingly discovered that, with the inventive culture media, the concentration of insulin in the culture media may be capable of being zero.

In another aspect, the present disclosure provides a method of using the culture media disclosed herein. In some cases, this is a method of making an engineered cell. In some cases, the culture media disclosed herein can be used for expansion or differentiation of cells. The cells may be used as food ingredients (e.g., cultured meat/seafood, or as supplements to add to plant-based meat products) or for pharmaceutical applications. Therefore, the culture media may be food-grade and/or pharmaceutical-grade. “Food-grade” may refer to products and/or compositions in any physical form which are intended to be consumed by human beings or lower animals in whole or part via the oral cavity. As used herein, the term “expansion” can refer to the increase in the number, density, or confluency of cells via replication, reproduction, etc., and the term “differentiation” can refer to the maturation and/or increased specialization of cells, as readily recognizable in the art. In some cases, the culture media disclosed herein can be used for expansion of muscle cells in a bioreactor, either on hollow fibers or microcarriers or in single-cell suspension or cell aggregate suspension (e.g., stirred-tank bioreactors, fluidized bed bioreactors, hollow-fiber bioreactors, rotating-wall bioreactors, wave bioreactors, packed-bed bioreactors, airlift bioreactors, etc.). In some cases, the culture media disclosed herein can be used for expansion of muscle cells for regenerative medicine applications (e.g., in the above-described bioreactors for the treatment of volumetric muscle loss). In certain cases, the culture media disclosed herein can be used as isolation media for generating primary muscle cell populations. These methods include incubating the cells in the serum-free cell culture media to allow expansion of the number of cells or allow for the differentiation of cells and the production of growth media (i.e. the serum-free cell culture media after support of cell growth). The cells may be incubated in any suitable device to support growth and/or differentiation of the cells and at a temperature, humidity and CO₂ concentration needed to support growth and/or differentiation of the cells. The cells and/or the growth media may be harvested by collecting both the cells and growth media and separating the growth media from the cells using any means available to those of skill in the art including use of centrifuges or allowing the cells to settle in a bioreactor, cell culture dish or other collection vehicle.

Prior to expanding the muscle cells, the method can further include either: i) coating a cell culture substrate or the cells with a cell adhesive peptide; or ii) adhering the cells to the cell culture substrate in the baseline serum-free and animal-component-free culture media and/or a different baseline serum-free and animal-component-free culture media that is lacking the plant protein composition. Methods of coating a cell culture substrate and adhering cells to the cell culture substrate are readily known in the art.

The cell adhesive peptide can be a recombinant version of a laminin (e.g., laminin 511) or fragments thereof, vitronectin or fragments thereof, poly-d-lysine, poly-l-lysine, fibronectin or fragments thereof, solubilized basement membrane or extracellular matrix preparation such as Matrigel, or other cell adhesive peptides understood by those skilled in the art. The cell adhesive peptides are suitably not derived from an animal but may be made recombinantly.

The adhering of step ii) can be performed in B8 culture media or other media that a skilled artisan would recognize as suitable for such adhering. Examples of other suitable media include essential 8 media, TeSR-E8 media, basal media (e.g., DMEM, DMEM/F12, etc.), proprietary serum-free media, serum-containing culture media, and other media understood by a skilled artisan to be suitable for adhering.

The cells discussed herein with respect to the culture media and the methods can be from an animal source, including, without limitation, from bovine, avian (e.g., chicken, quail), porcine, seafood, or murine sources. The cells are selected from the group comprising muscle satellite cells, fibroblasts, adipogenic precursor cells, mesenchymal stem cells, embryonic stem cells, and induced pluripotent stem cells. The cells may preferably be muscle satellite cells. The cells may preferably be bovine cells, or the cells may preferably be piscine cells. The cells and/or muscle satellite cells discussed herein with respect to the culture media and the methods can be derived from seafood such as fish (e.g., salmon, tuna, tilapia, perch, mackerel, cod, sardine, trout, etc.), shellfish (e.g., clams, mussels, and oysters); crustaceans (e.g., lobsters, shrimp, prawns, and crayfish), and echinoderms (e.g., sea urchins and sea cucumbers). The cells and/or muscle satellite cells discussed herein with respect to the culture media and the methods can be bovine, galline, ovine, porcine, equine, murine, caprine, lapine, or piscine. In some cases, the muscle satellite cells are bovine, galline, porcine, or piscine. The cells and/or muscle satellite cells may be immortalized, and immortalization can be obtained through genetic modification, spontaneous immortalization, or other means readily known in the art. The harvested cells may be used in a cultured food product, or to produce a food or pharmaceutical product. The growth media may also be used to produce a food or pharmaceutical product.

In another aspect, the present disclosure provides a method of making a serum-free and animal-component-free culture media. The method includes adding a plant protein composition to a basal culture media. The basal culture media may be serum-free and animal-component free. In some cases, this can involve mixing all of the components of the serum-free and animal-component-free culture media starting with water as a base material or alternatively a basal culture media may be used as the starting material. In some cases, certain portions can be pre-mixed before combining with other portions. A skilled artisan will recognize that the specific method of making the culture media of the present disclosure is not intended to be limiting to the protection of the culture media or the method of using the culture media. The plant protein composition is added to the basal culture media to a final concentration of 0.05 g/L to 2 g/L. The plant protein composition can be present in an amount of at least 50 mg/L, at least 100 mg/L, at least 200 mg/L, at least 250 mg/L, at least 500 mg/L, at least 750 mg/L, at least 1 g/L, at least 1.5 g/L, at least 2 g/L, at least 5 g/L, at least 10 g/L, or at least 15 g/L and at most 50 g/L, at most 25 g/L, at most 15 g/L, at most 10 g/L, at most 5 g/L, at most 2 g/L, at most 1.5 g/L, or at most 1 g/L. In some cases, the plant protein composition is present in an amount of about 200-1000 mg/L. The final media made via this method can be any of the media described herein.

The plant protein composition used in the serum-free cell culture media may be prepared by any means known to those of skill in the art including the means described herein in the Examples. For example, the plant protein composition used in the serum-free cell culture media may be prepared from a ground plant material, which may be a partially defatted or defatted plant material, including a plant cake material that is produced as a result of a plant oil processing system. The plant protein composition for use in the media may be prepared by alkali extraction, isoclectric precipitation of the alkali extract, and finally dissolution at physiological pH of the protein precipitate. The alkali extraction may comprise incubating a ground plant starting material in basic aqueous solution containing sodium hydroxide (for example, at pH 12.5) for up to twenty-four hours or more. The isoelectric precipitation may comprise adjusting the extract pH to acidic conditions (for example, pH 4.5) using hydrochloric acid for up to twenty-four hours or more. The dissolution of the precipitated proteins may comprise re-dissolving these precipitated proteins in basic aqueous solution containing sodium hydroxide (for example, pH 12.5) for up to twenty-four hours or more, optionally followed by adjusting the pH to physiological levels (about pH 7.2) using hydrochloric acid, for example. The resulting protein solution may be filtered and concentrated. Alkali conditions throughout can range from pH 8 to pH 13.5 and can be created with various bases (in addition to sodium hydroxide). Acidic conditions throughout can range from pH 3 to pH 6 and can be created with various acids (in addition to hydrochloric acid). Extraction may be preceded by incubating a ground plant starting material in a de-fatting solution comprising hexane, alcohol or other organic solvents, or a combination thereof. In some aspects, extraction is performed in solutions which are buffered with salts (such as sodium chloride, for example). Therefore, the plant protein composition used in the serum-free cell culture media may be prepared by alkali extraction followed by acid precipitation of an aqueous solution from a ground plant material, and the ground plant material may be defatted or partially defatted. The ground plant starting material may be ground whole plant material, such as ground whole soybeans, rapeseed, cottonseed or any of the plants disclosed herein or may be a defatted or partially defatted meal obtained after oil extraction from the plant material. The incubation may be in an alkali solution to aid in extracting and solubilizing the protein from the ground plant material. The protein may then be concentrated using means known to those of skill in the art such as centrifugation, acid (isoelectric) precipitation or ultrafiltration and the resulting plant protein composition which may be classified as a plant protein extract, concentrate or isolate depending on the starting material and the processing steps taken can be added to the media to obtain the concentration of plant protein in the final serum-free cell culture media desired.

Before discussing the exemplified aspects of the present disclosure, Applicant emphasizes that the inventiveness of the present disclosure lies heavily with the fact that the inventive culture media has been validated. Without wishing to be bound by a particular theory, we remain at the dawn of cultured meat products and it remains very challenging to predict efficacy, particularly when it relates to formulations that traditionally involve serum or animal products. Prior to this invention, to the best of Applicant's knowledge no successful culture media had been developed for long-term propagation of muscle satellite cells without the use of serum, other animal products, or non-food-safe components, aside from Applicant's own previous efforts involving a recombinant form of albumin. Additionally, prior to this invention, to the best of Applicant's knowledge no method for the passaging and propagation of these cells in a serum-free manor had been developed, aside from Applicant's own previous efforts involving a recombinant form of albumin. Additionally, prior to this invention, to the best of the Applicant's knowledge no method for replacing albumin with plant protein extracts had been developed for any cell culture, aside from Applicant's present work. As a result, the number of possibilities remains nearly infinite while the guidance from successful examples is practically zero. Because of this, Applicant submits that the bar for what might be considered inventive in this space needs to properly consider these factors.

No admission is made that any reference, including any non-patent or patent document cited in this specification, constitutes prior art. In particular, it will be understood that, unless otherwise stated, reference to any document herein does not constitute an admission that any of these documents forms part of the common general knowledge in the art in the United States or in any other country. Any discussion of the references states what their authors assert, and the applicant reserves the right to challenge the accuracy and pertinence of any of the documents cited herein. All references cited herein are fully incorporated by reference, unless explicitly indicated otherwise. The present disclosure shall control in the event there are any disparities between any definitions and/or description found in the cited references.

The following examples are meant only to be illustrative and are not meant as limitations on the scope of the invention or of the appended claims.

### Examples

After unsuccessful efforts described in the Comparative Examples below, in-house alternatives were explored using bulk proteins isolated from Inca Peanut (Plukenetia volubilis) protein powder, Soybean meal (Glycine max), Rapeseed meal (Brassica napus), and Cottonseed meal (Gossypium hirsutum) using simple alkaline extraction followed by acidic protein precipitation. This extraction yielded Inca Peanut Protein Isolate (IPPI), Soy Protein Isolate (SPI), Rapeseed Protein Isolate (RPI), and Cottonseed Protein Isolate (CPI). SDS-page analysis reveals that isolated proteins are primarily <100 kDa for all proteins, and <50 kDa for IPPI and RPI. Pierce-BCA protein quantification of the resulting solutions revealed isolate yield/starting material yields of ˜6.5 g/kg (IPPI), 188 g/kg (SPI), 39 g/kg (RPI), and 84 g/kg (CPI) after no process optimization. A rough estimate of cost/kg of IPPI, SPI, RPI, and CPI is given based on raw material input costs ($4,255, $1.75, $6.10, and $3.21, respectively), though actual costs are higher due to processing costs (e.g., NaOH and HCl used to adjust protein solution pH, and energy costs of centrifugation/filtration). However, it is clear that costs for all isolates are drastically lower than the >$40,000/kg for recombinant albumin, even including these simple processing steps.

### Example 1

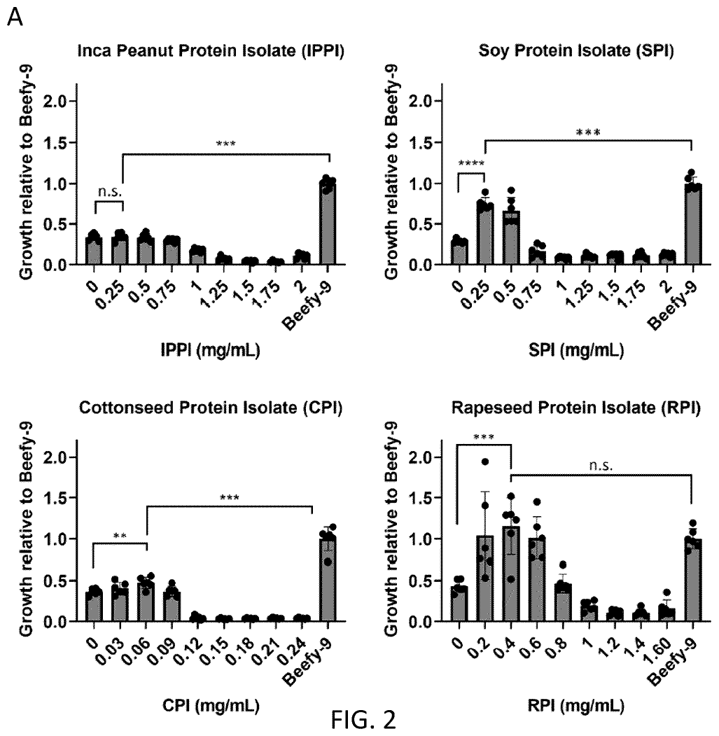

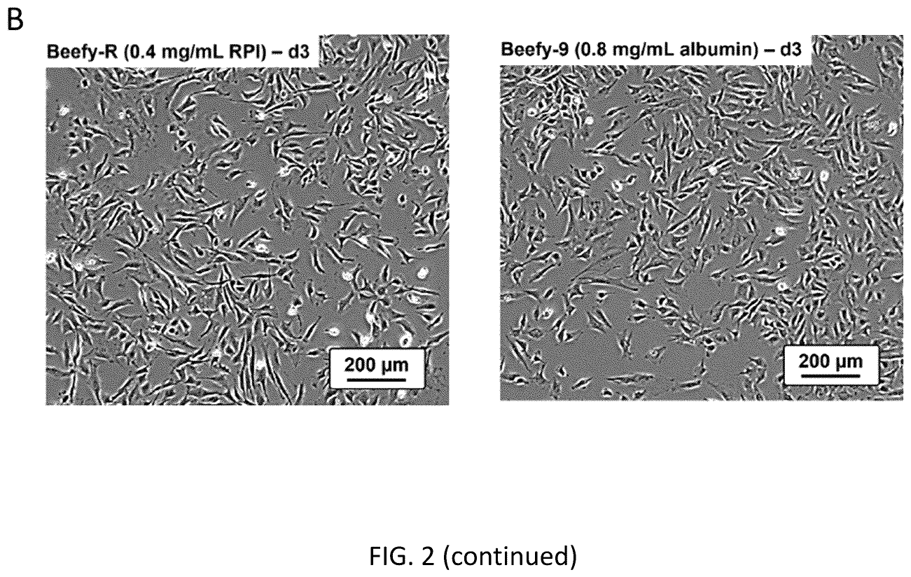

After generating protein isolates, BSC growth was analyzed over short-term (4 day) periods with different concentrations of the protein isolates (FIG. 2). Results show that IPPI does not improve growth, but that SPI, RPI, and CPI all do significantly improve growth compared with no supplement. Of these RPI offers the best growth, and indeed shows significant improvement over albumin-supplemented Beefy-9 at 0.4 mg/mL RPI. Along with growth kinetics, brightfield microscopy reveals no change in cell morphology when RPI is used instead of rAlbumin, further confirming that this can serve as a suitable low-cost replacement.

To overcome the high cost of albumin, we have explored a number of low-cost and animal-component free alternatives and have discovered that bulk protein extract from rapeseed meal (a byproduct of canola oil production) is particularly effective in replacing the albumin in Beefy-9 at a concentration of 0.4 mg/mL. We term this new media “Beefy-R,” and its composition can be found in Table 1 below:

TABLE 1
Beefy-R

| | | | Supplier (this | Catalog # (this |
|---|---|---|---|---|
| Component | Concentration | | work) | work) |
| DMEM/F12 (Basal media) | N/A | | ThermoFisher | 11330057 |
| 2-Phospho-L-ascorbic acid | 200 | ug/mL | Sigma | 49752-10G |
| trisodium salt | | | | |
| Insulin | 20 | ug/mL | Sigma | 91077C-250MG |
| Transferrin | 20 | ug/mL | ThermoFisher | 11107018 |
| Sodium selenite | 20 | ng/ml | Sigma | S5261-10G |
| FGF2 | 40 | ng/mL | Peprotech | 10018B1MG |
| NRG1 | 0.1 | ng/ml | VWR | 10770-630 |
| TGFb3 | 0.1 | ng/mL | R&D Systems | 8420B3005/CF |
| Rapeseed protein isolate (in- | 0.4 | mg/mL | In-house | N/A (Rapeseed |
| house) | | | (Rapeseed meal | meal ASIN |
| | | | from Amazon) | #B0006Q0EMW) |

Rapeseed and other (e.g., soy, yeast, wheat and rice) protein hydrolysates have previously been used as albumin replacements for other cell types (e.g., CHO cells), however, these supplements present additional costs in the form of hydrolytic enzymes or other processing to generate the hydrolysates. Therefore, the possibility of ultra-low-cost and simple protein isolates as albumin alternatives is a promising option for cultured meat.

To our knowledge, this is the first instance of rapeseed and other (i.e., soy and cottonseed) protein isolates without hydrolysis being used for animal cell culture media and as an albumin replacement, and the first demonstration of rapeseed protein isolates in any form being used for cell culture supplements for skeletal muscle cells (including bovine skeletal muscle cells) for any application (including cultured meat).

The high cost of cell culture media for cultured meat is the number one challenge facing the field's economic viability at scale. As such, there is a dramatic need for low-cost alternatives to canonical media components. Here, because cultured meat must ultimately compete with commodity agricultural products, it is important that inputs and processes are as simple, scalable, and low-cost as possible. Rapeseed protein isolate meets all of these needs. The global supply of rapeseed meal is massive, with 50 million metric tons produced annually as a byproduct of the canola oil production process. The use of this ingredient is therefore highly scalable. Furthermore, as a byproduct, rapeseed meal overs a highly sustainable input to the cultured meat production process, as it would require no further extraction of natural resources and would instead upcycle current waste-streams. Lastly, the production process of RPI is simple, scalable, and low-cost, using only alkaline and acid treatments as well as filtration and centrifugation. It is possible that alternative steps could be employed if desired to improve yields or reduce costs, such as dialysis and lyophilization instead of ultrafiltration to concentrate protein samples, or the use of filters rather than centrifugation to remove aggregates and particulates from protein solutions after acid precipitation and re-dissolution. Finally, it should be noted that in the context of cultured meat, another significant benefit of the described technology is the use of food-safe ingredients, including rapeseed proteins (which are used in human food products) and simple acids, bases, and salts. Ultimately, the significance of this work lies in 1) the ultra-low cost alternative to recombinant albumin, 2) the highly scalable nature of the process, and 3) the appropriateness of inputs and processes for use in a food-production system.

As mentioned, to our knowledge, this is the first instance of non-hydrolyzed rapeseed protein isolates in animal cell culture media and as an albumin replacement, the first use of rapeseed protein isolates in any form muscle stem cell culture, and the first use of rapeseed protein isolates for culture media in the context of cultured meat. The fact that rapeseed is more functional than the other proteins tested (FIG. 2) or than the other alternatives tested shows that this system is both novel and important. Further, the approach outlined here to screen non-hydrolyzed protein sources from agricultural or other sourced materials, suggests that additional substrates could be identified for low cost processes and supplements to use in cell cultivation processes, including not only muscle stem cells, but also adipose stem cells and differentiating cells.

### Comparative Example

Prior to even attempting the successful culture media described in Example 1, the inventors explored various compounds/compositions with the intention of attempting to replace the recombinant albumin from Beefy-9, due to its outsized role in the cost. The inventors first explored various compounds that might replace albumin's diverse roles in cell culture media, including as an as an antioxidant and carrier of numerous compounds, including fatty acids, ions, amino acids, signaling molecules, and other factors. Many of the compounds/compositions tested did not successfully replace albumin in Beefy-9, despite suggestion in the literature that these compounds would be suitable replacements for albumin in certain contexts.

The cyclodextrins α-cyclodextrin and β-cyclodextrin are often used as carrier molecules for numerous compounds (such as fatty acids in generating water-soluble lipid mixtures) and have been previously reported as substitutes for albumin in the serum-free culture of human fibroblasts and murine hybridoma cells. Similarly, other cyclodextrins (γ-cyclodextrin and hydroxypropyl-β-cyclodextrin) have been used in forming effective protein-free media for mouse myeloma cells. We explored these compounds as replacements for albumin in Beefy-9 but saw no improvement over B8 media with no albumin added (i.e., no ability to replace albumin function). With this data we ruled out α and β-cyclodextrin.

Other low-cost plant-derived components have also been tested as substitutes for albumin in cell culture media. Specifically, plant peptones (protein hydrolysates) from wheat and cotton were shown to effectively replace bovine serum albumin in culturing bovine embryos. Similarly, CHO cells have previously been cultured numerous plant hydrolysates (including rapeseed, soy, yeast, wheat, and rice) completely replacing proteins in culture media (e.g., transferrin, insulin, and albumin). We tested numerous supplier-provided hydrolysates, but again saw no ability to replace albumin.

Another potential role of albumin in the cell culture media is one of macromolecular crowding, due to its high concentration in the culture media, and the potential benefits of macromolecular crowding in cell culture media. We therefore tested polyethylene glycol (PEG) and dextran as macromolecular crowders. Dextran showed no improvement to cell growth, and while PEG showed some improvement at high concentrations, growth was still significantly less than Beefy-9 supplemented with albumin. As such, we determined that these molecules were ineffective albumin replacements.

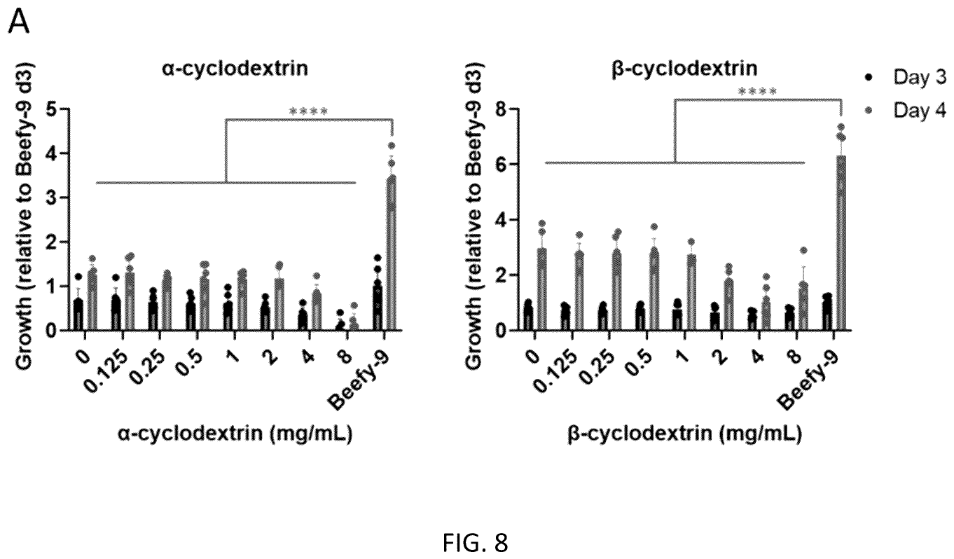

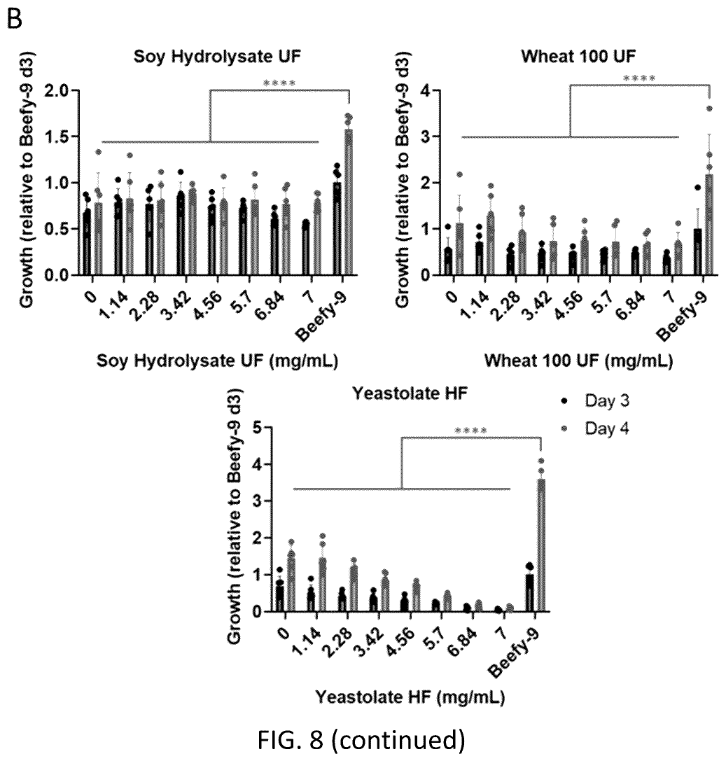

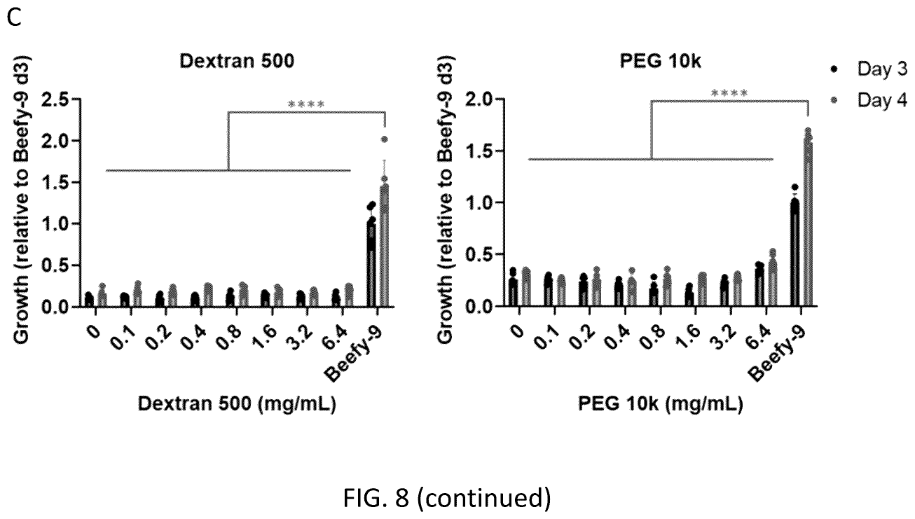

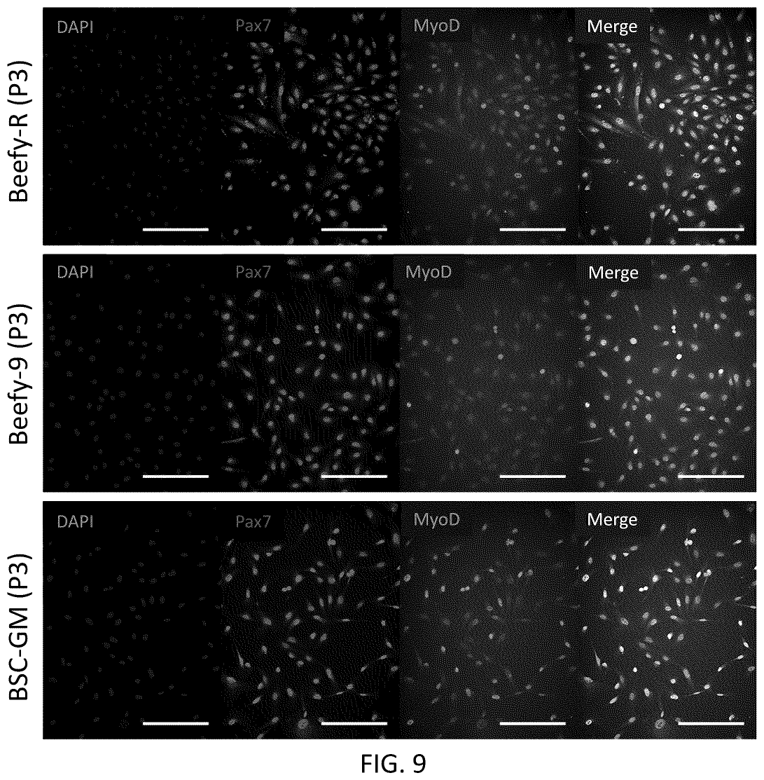

Some of the data for these unsuccessful formulations are included in FIG. 8. While some data have been excluded for brevity additional data can be provided to a patent examiner upon request.

Generally, the innovations described herein can possibly be used as an albumin alternative in lots of different serum-free media, including but not limited to B8/Beefy-9 media that are generally used as a scaffold in this application.

### Example 2: A Beefy-R Culture Medium: Replacing Albumin with Rapeseed Protein Isolates

The development of cost-effective serum-free media is essential for the economic viability of cultured meat. A key challenge facing this goal is the high-cost of recombinant albumin which is necessary in many serum-free media formulations, including a recently developed serum-free medium for bovine satellite cell (BSC) culture termed Beefy-9. Here we alter Beefy-9 by replacing recombinant albumin with rapeseed protein isolate (RPI), a bulk-protein solution obtained from agricultural waste through alkali extraction (pH 12.5), isoelectric protein precipitation (pH 4.5), dissolution of physiologically soluble proteins (pH 7.2), and concentration of proteins through 3 kDa ultrafiltration. This new medium, termed Beefy-R, was then used to culture BSCs over four passages, during which cells grew with an average doubling time of 26.6 h, showing improved growth compared with Beefy-9. In Beefy-R, BSCs maintained cell phenotype and myogenicity. Together, these results offer an effective, low-cost, and sustainable alternative to albumin for serum-free culture of muscle stem cells, thereby addressing a key hurdle facing cultured meat production.

### Introduction

Cultured meat (also cultivated or cell-based meat) is an emerging technology with the potential to overcome animal agriculture's limitations in terms of sustainability, animal welfare, and human health [1]. Briefly, cultured meat is produced through cell culture and tissue engineering, rather than by raising and slaughtering livestock. The potential benefits of cultured meat have been projected and discussed in several analyses and reviews [2-4]; however, substantial research and development is still required to make this technology viable at a scale and price-point comparable to conventional meat [5]. Here, a key area of research continues to be the culture media, or the liquid substrates that provide nutrients and functional factors (e.g., proteins) to cells to pro-mote growth and differentiation into relevant tissues (typically muscle and fat). Challenges to media development for cultured meat are a traditional dependence on animal-derived fetal bovine serum (FBS), or else the high cost of serum-free media formulations. As media are projected to account for a majority the costs of cultured meat, establishing affordable animal-free formulations is essential for achieving price parity with conventional meats [6].

Recently, we developed an animal-component free medium that is suitable for growing bovine satellite cells (BSCs) [7]. This medium, termed Beefy-9, was developed by adding recombinant albumin to a previously developed medium (B8), which had itself been established for culturing induced pluripotent stem cells (iPSCs) [8]. While the addition of recombinant albumin rendered this medium suitable for BSCs, it also added substantial cost. Specifically, depending on its con-centration, albumin addition resulted in a 50-400% increase in cost compared with B8. This price-dominance of albumin has been noted by others as well [9]. As such, it is clear that cheaper alternatives must be developed to bring down the cost of serum-free media.

One promising option is to use proteins extracted from low-cost and abundant sources. For instance, hydrolyzed proteins from rapeseed, wheat, soy, chickpeas, and other plants or agricultural byproducts have been used as albumin alternatives for culturing Chinese hamster ovary (CHO) and other mammalian cell types [10-15]. These sources offer the benefits of being low-cost, scalable, and obtained from byproducts of agricultural processes (such as the production of canola oil in the case of rapeseed). However, while the hydrolysis used in these studies can enable the production of well-defined peptide fractions with specific functionalities, it can also add complexities (e.g., hydrolytic enzymes or chemical treatments) which may impact the cost and scalability of the inputs. Thus, it may be valuable to use bulk plant protein extracts (i.e., nonhydrolyzed) to maximize simplicity, affordability, and scalability.

The current work demonstrates that bulk protein extracts from oilseed protein meals can serve as albumin alternatives in Beefy-9. Specifically, rapeseed protein isolate (RPI) produced through simple alkali extraction and isoelectric precipitation can fully replace albumin in BSC culture over short- and long-term growth, thereby dramatically lowering the cost of the medium. More, this modified serum-free medium (termed Beefy-R) supports enhanced cell growth compared with Beefy-9, while maintaining the phenotype and differentiation capacity of BSCs at least as well as Beefy-9. Together, these findings demonstrate the utility of oilseed protein isolates (OPIs), and particularly RPI, in replacing albumin in cell culture, thereby enabling substantial cost reduction for cultured meat.

### Materials and Methods

### Bovine Satellite Cell Culture and Routine Maintenance

Primary bovine satellite cells (BSCs) used in this study were isolated and characterized previously using tissue from a 2-week old Simmental calf [7]. For routine cell maintenance, cells were cultured in 37° C. with 5% CO₂ on tissue-culture plastic coated with 0.25 μg/cm² iMatrix recombinant laminin-511 (Iwai North America #N892021, San Carlos, CA, USA). BSC growth media (BSC-GM) was used, comprised of DMEM Glutamax (ThermoFisher #10566024, Waltham, MA, USA), 20% fetal bovine serum (FBS; ThermoFisher #26140079), 1 ng/mL human FGF-2 (ThermoFisher #68-8785-63), and 1% antibiotic-antimycotic (ThermoFisher #1540062). For routine culture, cells were expanded to a maximum of 70% confluence, harvested using 0.25% trypsin-EDTA (ThermoFisher #25200056), counted using an NC-200 automated cell counter (Chemometec, Allerod, Denmark) and either seeded at a density of 2,000-5,000 cells/cm² on new tissue culture plastic with growth medium and laminin, or else frozen in FBS with 10% Dimethyl sulfoxide (DMSO, Sigma #D2650, St. Louis, MO, USA).

### Oilseed Protein Isolate Generation

Oilseed protein isolates (OPIs) were generated using protein meals from four plant sources. These were Inca peanut (Plukenetia volubilis), which was obtained as a protein powder (Imlak′esh Organics #765857605340, Santa Barbara, CA, USA); soybean (Glycine max), which was obtained as ground protein meal (Roots organics #715110, Eugene, OR, USA); rapeseed (Brassica napus), which was obtained as protein cakes (Joshua Roth Limited #6046, Albany, OR, USA); and cottonseed (Gossypium hirsutum), which was obtained as ground protein meal (Down To Earth #07859, Eugene, OR, USA). Rapeseed protein cakes were ground in a standard coffee grinder to generate a ground protein meal, after which protein isolation procedures were the same for all samples, based on adapted methods from several previously reported studies [10,16-23]. Briefly, for each protein, protein meal was suspended in DI water (10% w/v), and the resulting slurry was adjusted to a pH of 12.5 using 5 M NaOH. The slurry was mixed at room temperature in a beaker with a magnetic stir bar for 1 h to extract proteins. Next, the mixture was centrifuged at 15,000 g and room temperature for 10 min to pellet non-soluble components, and the protein-containing supernatant was collected. This supernatant was adjusted to pH 4.5 with 6 M HCl in order to precipitate out proteins, and solution was again centrifuged at 15,000 g and room temperature for 10 min. The supernatant was dis-carded, the protein-containing pellet was resuspended in the same volume of DI water used previously, and the mixture was adjusted to pH 12.5 using 5 M NaOH. Proteins were encouraged to redissolve and redisperse in alkali solutions using a magnetic stir-bar and mechanical disruption of the pellets. When proteins were completely dispersed, pH was adjusted to 7.2 using 6 N HCl in order to crash out those proteins that were not soluble at physiological pH. This mixture was centrifuged at 15,000 g and room temperature for 30 min, and the supernatant was filtered through a 10 μm filter before being centrifuged at 45,000 g and 4° C. for 3 h to completely pellet any insoluble particles. The resulting OPI-containing supernatant was collected, filtered through an 0.22 μm filter, and concentrated with 3-kDa cutoff ultrafiltration tubes (Pall #MAP003C37, Port Washington, NY, USA) by centrifugation at 4,500 g and 4° C. until 50- to 100-fold concentration had been achieved.

Concentrated proteins were filtered through 0.45 μm filters (Sigma #SLHV033R) and were then quantified using a Pierce BCA kit (ThermoFisher #23227) according to the manufacturers protocol. Once protein concentrations had been determined, they were adjusted to 50 mg/mL for all samples using sterile ultrapure water, and aliquots were snap-frozen in liquid nitrogen before moving them to long-term storage at 80° C. Proteins could be thawed to room temperature without any impact on solubility, though long-term storage at 4° C. or lyophilization resulted in aggregation.

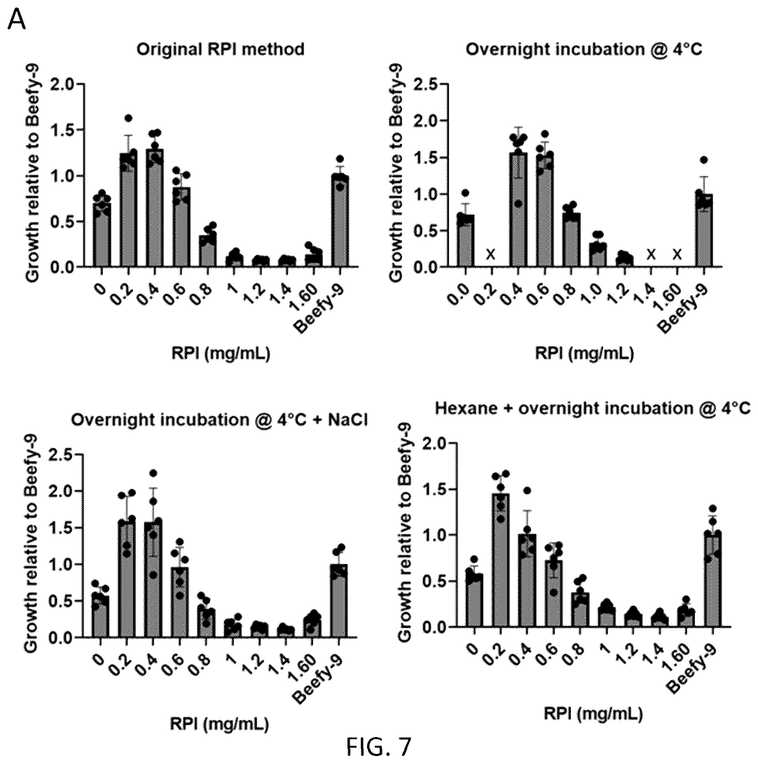

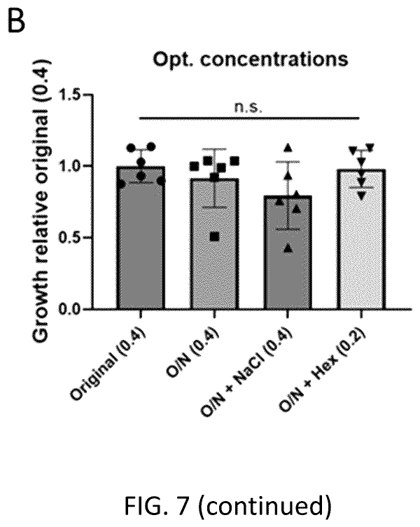

To test how different protein isolation methods affected growth (FIG. 7), the following adjustments to the protocol were made: first, an overnight incubation at 4° C. was added between the 10 μm filter step and the centrifugation at 45,000 g. Second, this same overnight incubation was added, but the protein solution was also supplemented with 120.6 mM NaCl. Third, hexane-defatting was performed before extraction by adding protein meals to hexane (Sigma #270504-1 L) (10% w/v) and incubating the mixtures at room temperature on a stir-plate for 1 h. After defatting, hexane was removed by decanting and protein meals were allowed left in the fume hood until fully dry. Defatted protein meals were then used for extraction as mentioned above, including the overnight incubation at 4° C. between 10 μm filtration and 45,000 g centrifugation. As noted in the results, no significant differences were noticed between these changes, except that hexane defatted RPI showed optimal performance at a reduced concentration. It should also be noted that preliminary experiments for this study only used centrifugation up to 12,000 g for all steps and still saw RPI efficacy. It is therefore expected that lower-speed centrifugation will work in settings that do not have large, high-speed centrifuges, though times may need to be increased to achieve sufficient pelleting to avoid clogging filters.

### Oilseed Protein Isolate Characterization: SDS-PAGE.

For initial protein characterization, an SDS-Polyacrylamide gel (SDS-PAGE) was generated using the Laemmli protocol. Briefly, 120 μg of each OPI solution was diluted in 50 μL of water and mixed with 10 μL of 6× Laemmli reducing buffer (ThermoFisher #J61337-AC). Solutions were heated to 95° C. for 10 min, centrifuged at 13,000 g for 5 min, and 10 μL of solution was loaded into a 4-20% Tris-glycine gel (ThermoFisher #XP04200PK2) along with 3 μL of Precision Plus protein ladder (Bio-Rad #1610374), and gels were run at 125 V for 1.25 h in Tris-Glycine running buffer (ThermoFisher #LC2675).

### Oilseed Protein Isolate Growth Studies

Hi-Def B8 medium was prepared by adding Hi-Def B8 aliquots (Defined Bioscience #LSS-201, San Diego, CA, USA) to DMEM/F12 (ThermoFisher #11320033) along with 1% antibiotic-antimycotic. Beefy-9 medium was prepared by adding 0.8 mg/ml of recombinant albumin (Sigma #A9731-1G) to Hi-Def B8. Next, Hi-Def B8 medium supplemented with various concentrations of OPIs or other supplements (FIG. 8) were prepared. Cells were thawed into 96-well plates in BSC-GM with 0.25 μg/cm² of recombinant laminin-511 and at a cellular density of 2,500 cells/cm². Cells were allowed to adhere overnight in serum-containing media in order to ensure consistent starting cell numbers, after which cells were washed once with DPBS (ThermoFisher #14190250) and media was changed to either B8, B8 supplements (e. g., OPIs), or Beefy-9. Two replicate plates were generated for each experiment, one of which was imaged, aspirated and frozen at −80° C. on day three, and one of which had its media refreshed on day three and was then aspirated and frozen at 80° C. on day four for dsDNA quantification. This quantification was performed using a FluoReporter dsDNA quantification kit (ThermoFisher #F2962) according to the manufacturer's instructions. Fluorescence was read on a Synergy H1 microplate reader (BioTek Instruments, Winooski, VT, USA) using excitation and emission filters centered at 360 and 490 nm. Fluorescence was analyzed relative to the Beefy-9 positive control and used to compare four-day growth between conditions.

### Multi-Passage Growth Studies

Once short-term growth had been assessed, multi-passage growth studies were performed to further validate the utility of RPI-supplemented Beefy-R medium. These studies used methods previously established in the development of Beefy-9 [7]. Briefly, BSCs (passage 2) were seeded into triplicate wells of 6-well plates (Corning #353046, Corning, NY, USA) in BSC-GM with 0.25 μg/cm² iMatrix laminin-511. After allowing cells to adhere overnight, cells were washed 1× with DPBS and fed either BSC-GM, Beefy-9, or Beefy-R (Hi-Def B8 supplemented with 0.4 mg/mL RPI). Beefy-9 and Beefy-R were prepared immediately before use. For passaging, cells were cultured to 70% confluency, rinsed 1× with DPBS, and dissociated with 500 μL of TrypLE Express (ThermoFisher #12604021). After incubating cells at 37° C. for 10 min, plates were vigorously tapped to dislodge cells, and cells were collected with an additional 1.5 mL of either BSC-GM or Hi-Def B8, depending on whether they were the serum-containing or serum-free samples. Cells were then counted using an NC-3000 automated cell counter (Chemometec), centrifuged at 300 g for 5 min, resuspended in either BSC-GM or Hi-Def B8, re-counted, and seeded onto new 6-well plates with 0.25 μg/cm² of iMatrix laminin-511 for BSC-GM cells, or 1.5 μg/cm² of truncated recombinant human vitronectin (Vtn-N; ThermoFisher #A14700) for serum-free cells. After allowing cells to adhere overnight, media was replaced with BSC-GM, Beefy-R, or Beefy-9, as appropriate. This process was repeated over 13 days and four passages (P2-P5) with cell feeding every two days. As before, Beefy-R and Beefy-9 were prepared immediately before use. When seeding passage three, additional wells were seeded for qPCR and immunostaining analyses.

### Serum-Free Differentiation and Phenotype Analysis

A population of cells at passage three was cultured to confluency in appropriate media, at which point media was changed to a previously described serum-free differentiation medium containing Neurobasal (Invitrogen #21103049, Carlsbad, CA, USA) and L15 (Invitrogen #11415064) media in a 1:1 ratio, supplemented with 10 ng/mL insulin-like growth factor 1 (IGF-1; Shenandoah Biotechnology #100-34AF-100UG, Warminster, PA, USA), 100 ng/mL epidermal growth factor (EGF; Shenandoah Biotechnology #100-26-500UG), and 1% antibiotic-antimycotic [24]. Cells were differentiated for 2 days before performing relevant analysis.

### Gene Expression Analysis

To assess relative gene expression between BSCs cultured in various media types, qPCR was performed on proliferative (70% confluency) and differentiated cells from P3 following standard protocols. Briefly, RNA was harvested using an RNEasy Mini kit (Qiagen #74104, Hilden, Germany) and cDNA was prepared with an iScript cDNA synthesis kit (Bio-Rad #1708890, Hercules, CA, USA) using 1 μg of RNA for each reaction. Next, qPCR was performed using 2 μL of cDNA and 1× TaqMan Fast Universal PCR Master Mix without AmpErase UNG (ThermoFisher #4352042). Primers used in this study were: 18 S (ThermoFisher #Hs03003631), Pax3 (ThermoFisher #Bt04303789), MyoD1 (Thermo-Fisher #Bt03244740), Myogenin (ThermoFisher #Bt03258929), and Myosin Heavy Chain (ThermoFisher #Bt03273061). Reactions were performed according to the manufacturer's instructions on a Bio-Rad CFX96 Real Time System thermocycler, and results were analyzed as 2^{-ΔΔCt} normalized to expression of the 18 S housekeeping gene and analyzed relative to proliferative BSC-GM expression for each gene.

### Immunostaining

Along with qPCR, immunocytochemistry was performed to assess phenotype of both proliferative and differentiated cells from each media. Cells were fixed with 4% paraformaldehyde (ThermoFisher #AAJ61899AK) for 30 min, washed 3× in DPBS, and permeabilized for 15 min with 0.5% Triton-X (Sigma #T8787) in DPBS. Cells were then rinsed 3× in PBS-T (DPBS containing 0.1% Tween-20 [Sigma #P1379]) and blocked for 45 min using a blocking buffer containing DPBS with 5% goat serum (ThermoFisher #16210064) 0.05% sodium azide (Sigma #S2002) before adding primary antibodies and incubating overnight at 4° C. For proliferative cells, primary antibodies used were for Pax7 (ThermoFisher #PA5-68506) diluted 1:500 in blocking buffer and MyoD (ThermoFisher #MA5-12902; 1:100). For differentiated cells, primary antibodies used were for MHC (Developmental studies hybridoma bank #MF-20, Iowa City, IA, USA; 4 μg/mL) and Phalloidin-488 (Abcam #ab176753, Cambridge, UK; 1:1000). Following primary antibody incubation, cells were rinsed 3× in PBS-T, incubated for 15 min at room temperature in blocking buffer, and treated with secondary antibodies in blocking buffer for 1 h at room temperature. Secondary antibodies for proliferative cells were anti-rabbit (ThermoFisher #A-11072; 1:500) and anti-mouse (ThermoFisher #A-11001) for Pax7 and MyoD, respectively, as well as a DAPI nuclear stain (Abcam #ab104139, Cambridge, UK; 1:1000). Secondary antibodies for differentiated cells were anti-mouse (ThermoFisher #A-11005) for MHC, as well as a DAPI nuclear stain. Finally, cells were rinsed 3× with DPBS before imaging.

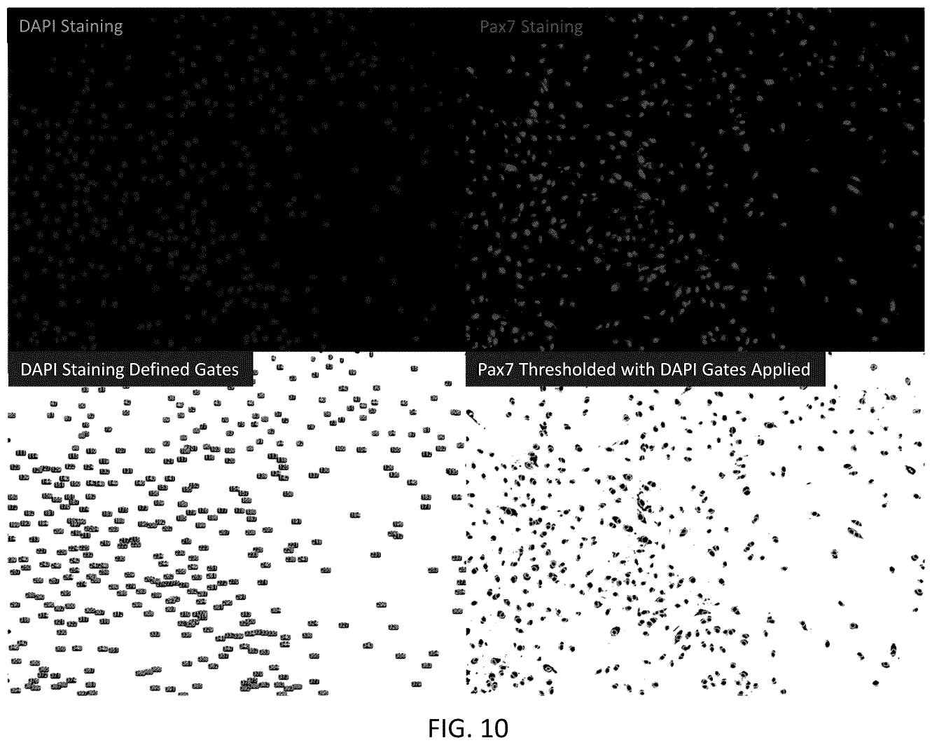

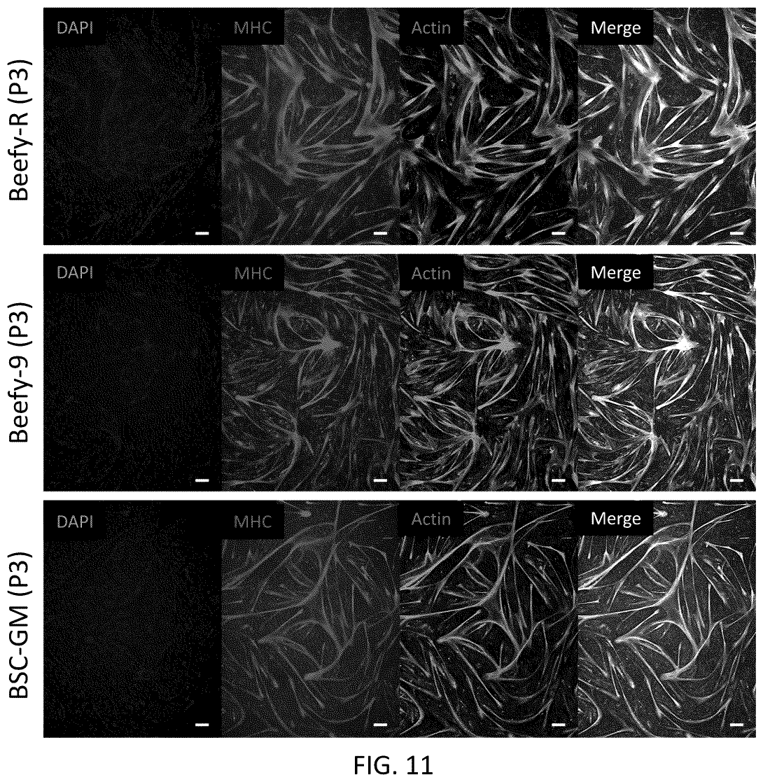

Imaging was performed with a KEYENCE BZ-X810 fluorescent microscope (Osaka, Japan). For Pax7 quantification and fusion index analyses, batch images were taken with the 10 objective at random points of culture wells selected by the KEYENCE software. Images were analyzed using ImageJ software. Briefly, for Pax7 quantification, each nucleus in the DAPI channel was established as a discrete region of interest (ROI), and these ROIs were added to the Pax7 channel after thresholding at a consistent value. Percentage of ROIs containing Pax7 signal was recorded (FIG. 10). For fusion index quantification, total nuclei were counted in the DAPI channel, after which MHC channels were used to generate nuclear selections following the application of a consistent threshold. Nuclei within the selections were counted, and the ratios of selected nuclei to total nuclei were recorded as fusion indices.

Growth Studies Using RPI from Multiple Lots of Rapeseed Protein Cakes

To test how variability in starting materials affected performance of Beefy-R, two different preparations of RPI were prepared as before. These included Lot #1, which was the same lot of rapeseed protein cakes used previously (sourced by Joshua Roth Limited in 2021), and Lot #2, using new rapeseed protein cakes (sourced by Joshua Roth Limited in 2022). Short-term screens of these two RPIs were performed as before but using the CyQUANT cell proliferation assay (ThermoFisher #C7026) for final assessment according to the manufacturer's instructions. Long-term growth-curves were generated with the same methods as before and using Beefy-9, Beefy-R containing 0.2 mg/mL of RPI (Lot #1), or Beefy-R containing 0.2 mg/mL of RPI (Lot #2). Both short-term and long-term growth experiments were then performed using immortalized bovine satellite cells (iBSCs) which were developed previously [25]. These cells (P47 at the time of this study) were maintained similarly to primary BSCs, but with additional media supplementation with 2.5 μg/mL puromycin (ThermoFisher #A1113803).

### Proteomics

Proteomic analysis was performed at the Massachusetts Institute of Technology's Koch Institute Proteomics core. First, Proteins were reduced with 10 mM dithiothreitol (Sigma) for 10 min at 95° C. and then alkylated with 20 mM iodoacetamide (Sigma) for 30 min at 25° C. in the dark. Proteins were than digested with trypsin on S-trap micro columns (Protifi #C02-micro-80) per the manufacturer's instruction. Next, the tryptic peptides were separated by reverse phase HPLC (Thermo Ulti-mate 3000) using a Thermo PepMap RSLC C18 column (2 um tip, 75 um×50 cm #ES903) over a 100-min gradient before nanoelectrospray using an Exploris mass spectrometer (Thermo). Solvent A was 0.1% formic acid in water and solvent B was 0.1% formic acid in acetonitrile. The gradient conditions were 1% B (0-10 min at 300 nL/min), 1% B (10-15 min, 300 nL/min to 200 nL/min), 1-7% B (15-20 min, 200 nL/min), 7-25% B (20-54.8 min, 200 nL/min), 25-36 B (54.8-65 min, 200 nL/min), 36-80% B (65-65.5 min, 200 nL/min), 80% B (65.5-70 min, 200 nL/min), 80-1% B (70-70.1 min, 200 nL/min), 1% B (70.1-90 min, 200 nL/min). The mass spectrometer was operated in a data-dependent mode. The parameters for the full scan MS were: resolution of 120,000 across 375-1600 m/z and maximum IT 25 ms. The full MS scan was followed by MS/MS for as many precursor ions in a 2 s cycle with a NCE of 28, dynamic exclusion of 20 s and resolution of 30,000.

Raw mass spectral data files (.raw) were searched using Sequest HT in Proteome Discoverer (Thermo). Sequest search parameters were: 20 ppm mass tolerance for precursor ions; 0.05 Da for fragment ion mass tolerance; 2 missed cleavages of trypsin; fixed modification were car-bamidomethylation of cysteine and TMT 10-plex modification on the lysines and peptide N-termini; variable modifications were methionine oxidation, tyrosine, serine and threonine phosphorylation, methionine loss at the N-terminus of the protein, acetylation of the N-terminus of the protein and also Met-loss plus acetylation of the protein N-terminus. For peptide groups data only PSMs with a Xcorr score greater than 2, isolation interference less than or equal to 30 and a deltaM (ppm) between 3 and 3 were used. For Gene Ontology (GO) analysis of the data, gene ontology annotation (GOA) files from UniProt were referenced in Scaffold (Proteome Software, Inc., Portland, OR; version 4.7.3). The GOA files used for CPI, SPI, and RPI were 4102840. G_hirsutum_U-pland_cotton_Gossypium_mexicanum.goa, 23,297. G_max.goa, and 388,940. B_napus_Rape.goa, respectively. Normalized Spectral Abundance Factors (NSAFs) of the GO terms in each of the three branches (molecular function, biological process, and cellular component) were averaged between three technical replicates for each OPI and used for GO representation. These NSAFs were calculated as described previously [26,27].

### Statistical Analysis

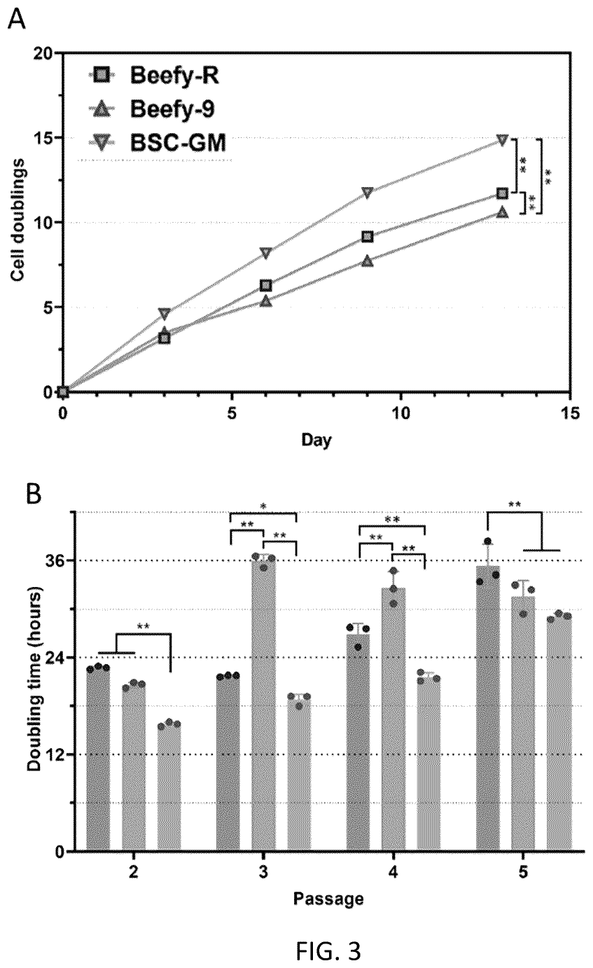

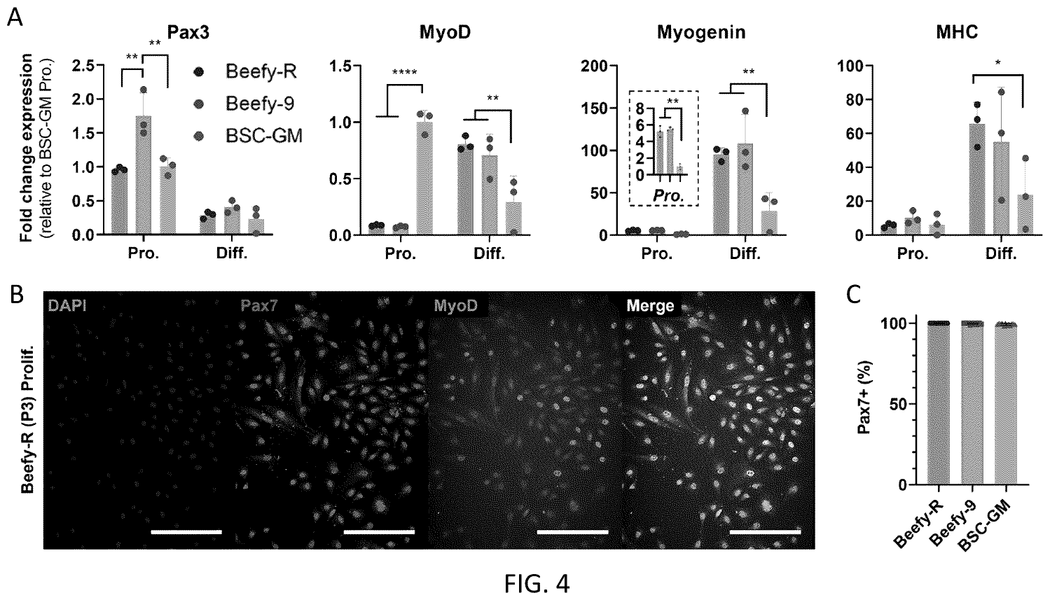

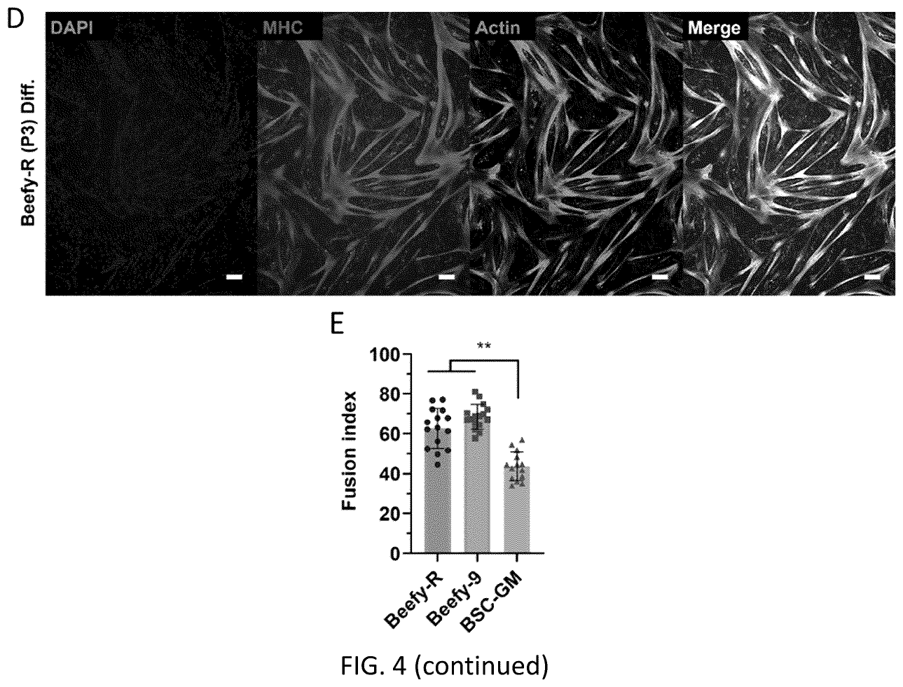

GraphPad Prism 9.0 software (San Diego, CA, USA) was used to perform all statistical tests. Specifically, for short-term growth studies, multiple t-tests were performed between B8 controls and optimal OPI-supplemented media, and between optimal OPI-supplemented media and Beefy-9 controls. A Bonferroni correction was used to adjust for two comparisons (adjusted α 0.025). For short-term growth studies be-tween different lots of rapeseed protein cakes, multiple t-tests were performed where, for each lot of RPI, tests were performed between B8 controls and media supplemented at 0.2 mg/mL, and between the two different lots of RPI at 0.2 mg/mL. Bonferroni corrections were used to adjust for two comparisons (α=0.025). For multi-passage analyses with SpC_{N}/L_{N} primary BSCs, two-way ANOVA was performed along with a Tukey's HSD post-hoc test between all samples of a certain timepoint, and statistical significance was indicated for comparisons at the final timepoint only (FIG. 3a) or for comparisons at all timepoints (FIG. 3b). For qPCR studies, two-way ANOVA was performed with a Tukey's HSD post-hoc test between all samples of a certain group (Pro. Or Diff.), and statistical significance was indicated (FIG. 4a) for all comparisons. For fusion index studies, a one-way ANOVA was performed along with a Tukey's HSD post-hoc test between samples (15 images per condition, except in the case where imaging artifacts clearly impacted image quality), and statistical significance was indicated (FIG. 4c) for all comparisons. P values <0.05 were treated as statistically significant, and all errors are given as ±standard deviation.

### Results

### Alkali Extraction of Protein Meals Enables Simple and Low-Cost Generation of Protein Isolates

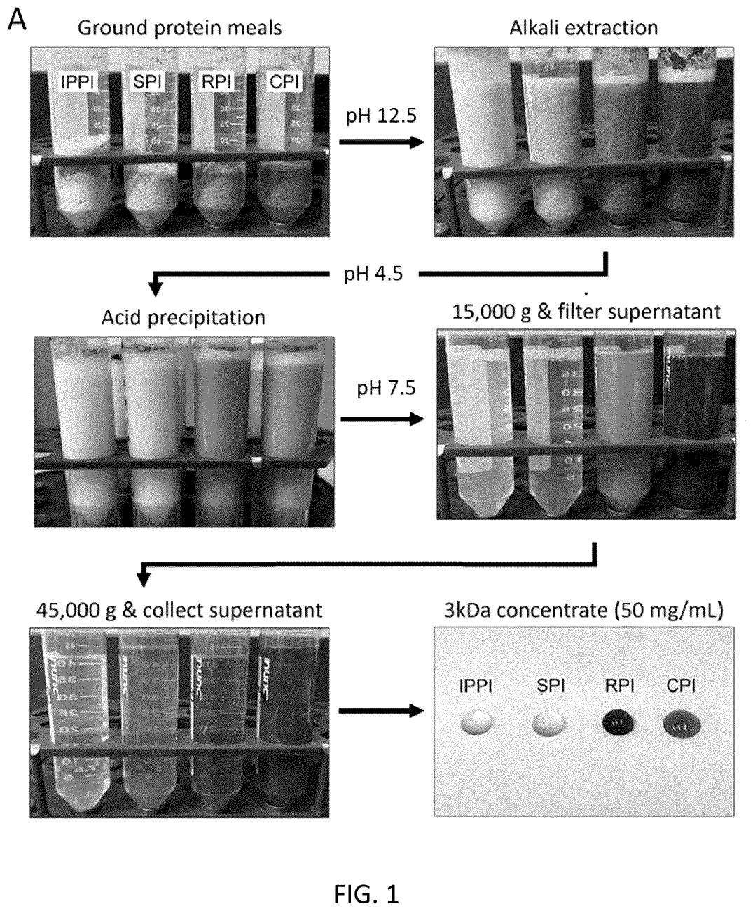

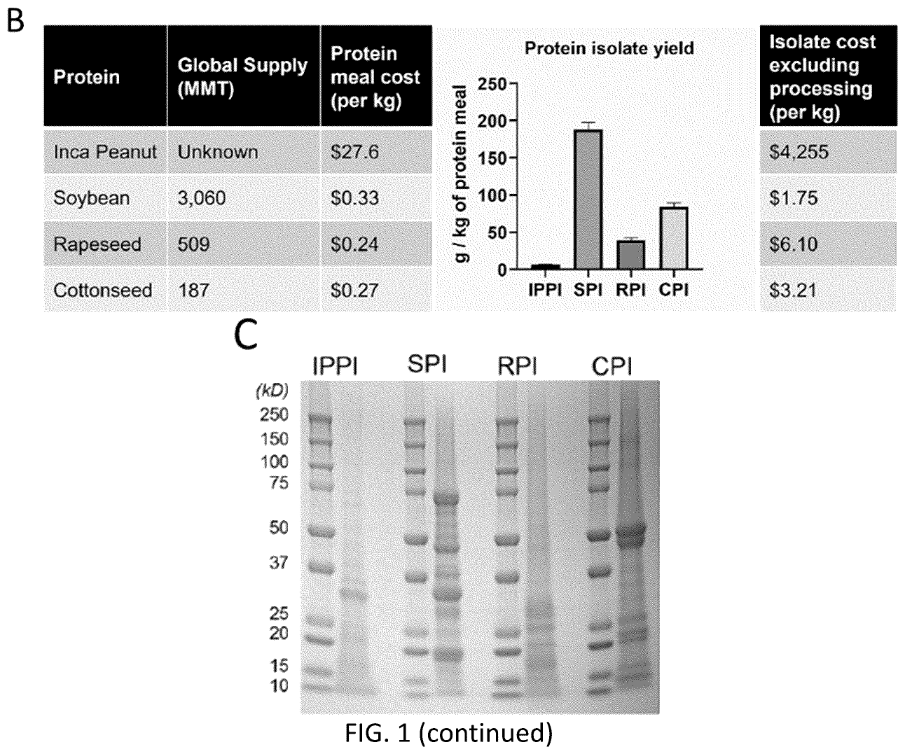

Oilseed protein isolates (OPIs) from four sources were used in this study: Inca peanut (Plukenetia volubilis), soybean (Glycine max), rapeseed (Brassica napus), and cottonseed (Gossypium hirsutum). OPIs were pre-pared using the same methods for each source, and involved alkali extraction (pH 12.5), isoelectric precipitation (pH 4.5), centrifugation (45,000 g), and filtration (3 kDa). This resulted in Inca peanut protein isolate (IPPI), soy protein isolate (SPI), rapeseed protein isolate (RPI), and cottonseed protein isolate (CPI). The isolates were either clear in color (IPPI and SPI) or reddish brown (RPI and CPI) and were slightly viscous (FIG. 1a).

Following extraction, OPI yields were analyzed against starting material weight to reveal isolation yields of 6.5 g/kg, 188 g/kg, 39 g/kg, and 85 g/kg for IPPI, SPI, RPI, and CPI, respectively (FIG. 1b). It is likely that optimizing extraction methods could enhance yields. Indeed, numerous studies have shown how changing protein meal preparation, defatting procedure, alkali extraction pH, isoelectric precipitation pH, ultrafiltration molecular weight cutoff, and other metrics can affect isolate yields and compositions [21,23,29-31]. As pertinent examples, studies with rapeseed protein isolates have shown significant yield improvements by precipitating proteins at higher temperature (50° C.) as opposed to room temperature used in this study, or via sequential iso-electric precipitation steps [32,33]. NSAF_{N}=Σn (SpCᵢ/Lᵢ)

Similarly, studies with soy protein isolates have shown improved yields of physiologically-soluble proteins by precipitating at a pH of 5.5, rather than 4.5 used in this study [34] where N is the protein index, SpC_{N} is the spectral count associated with protein N, L_{N} is the amino acid length of protein N, and n is the total number of proteins assessed for a given OPI. In other words, NSAF_{N} represents the Spectral count of protein N divided by the length of protein N, normalized to the sum of all SpC/L for all proteins.

The mass spectrometry proteomics data have been deposited to the ProteomeXchange Consortium via the PRIDE partner repository with the dataset identifier PXD036518.

Finally, studies with cottonseed protein isolates have shown increased yields with a 90 min extraction as compared with the 60 min used in this study 30. These and other changes can be explored to optimize protein yields, as well as exploring wholly other advanced methods of protein isolate preparation [35]. While the Inca peanut protein powder was originally produced for food and therefore relatively expensive ($27.6/kg), the three other protein meals were the byproduct of food oil production and are therefore attainable at low cost (less than $0.4/kg). The annual global supply of soybean meal, rapeseed meal, and cottonseed meal is 3,060, 509, and 187 million metric tons, respectively [36]. From measured yields (FIG. 1b), raw-material input costs of OPIs for IPPI, SPI, CPI, and RPI were $4255/kg, $1.75/kg, $6.10/kg, and $3.21/kg, respectively. These calculations do not include processing costs (e.g., solutions, electricity, filters, etc.), and so real-world cost-of-goods would be higher; however, even accounting for processing expenses, the costs of SPI, RPI, and CPI are likely to be orders of magnitude lower than the cost of recombinant albumin available today, which is around $45,000/kg (Cellastim S, InVitria). This suggests that OPIs could be affordable albumin alternatives. There is also likely room for process optimization to reduce costs further.

Finally, following protein isolation, basic characterization was performed via SDS-PAGE (FIG. 1c). The results showed heterogeneous mixtures of proteins which were <75 kDa for all OPIs. SPI and CPI showed higher levels of large proteins (>37 kDa), while the majority of proteins in IPPI and RPI were <37 kDa. Observed proteins likely correspond with fractions of various albumins and globulins, which are the largest fractions of seed storage proteins, and which exist in the size ranges observed [23,37-41]. Specifically, albumins from OPIs exist around ˜25-35 kDa for inca peanut [37], ˜20-35 kDa for soybean [42], ˜10-20 kDa for cottonseed [43], and ˜10-15 kDa for rapeseed [29]. Globulins from these seed proteins exist around ˜ 10-40 kDa for inca peanut [23], ˜20-70 kDa for soybean [44], ˜20-60 kDa for cottonseed [43], and ˜15-50 kDa for rapeseed [29]. Ultimately, these results point towards heterogeneous and varied mixtures of proteins in all four OPIs.

### Oilseed Protein Isolates can Replace Albumin in Serum-Free Media for Short-Term Growth

The bovine satellite cells (BSCs) used for this work have been extensively characterized and validated in previous studies [7,45]. To test the ability of OPIs to replace albumin in serum-free media, BSCs were cultured for four days in either B8 without supplementation, B8 with OPI supplementation at a range of concentrations, or Beefy-9 (B8 0.8 mg/mL recombinant albumin). The results showed that SPI, CPI, and RPI significantly improved cell growth compared with B8, while IPPI did not (FIG. 2a). Here, both SPI and RPI performed better than CPI, while RPI at 0.4 mg/mL performed the best of all supplements and completely recovered the efficacy of albumin (1.15-fold efficacy compared with Beefy-9). From these results, a new medium termed Beefy-R was established which comprises B8 with 0.4 mg/mL RPI. Cells in Beefy-R showed no morphological differences from those in Beefy-9 (FIG. 1b). Interestingly, all OPIs showed growth inhibition at higher concentrations. This is in contrast to recombinant albumin, which did not exhibit inhibitory effects during the development of Beefy-9 [7]. This suggests that there exists some other growth-inhibiting or cytotoxic component(s) of OPIs which negatively affect cell growth above a threshold concentration. Future work to identify and remove these inhibitory components would be valuable for OPI optimization.

To briefly explore how extraction methods impact RPI's efficacy, isolates were prepared using altered methods, including pre-extraction hexane defatting, increased salt concentrations, and protein extraction with or without an overnight incubation at 4° C. before final concentration steps. No significant difference was seen between optimum concentrations of the various methods (FIG. 7), and so the original method was used. To compare the efficacy of RPI with other potential albumin alternatives, short-term experiments were performed using B8 supplemented with candidates which had previously been demonstrated as albumin alternatives in other culture systems, including cyclodextrins [46,47], off-the-shelf plant protein hydrolysates [48], and molecular crowders [49]. None of these proved effective at the concentrations tested (FIG. 8), suggesting the unique potential of RPI in serum-free BSC culture.

Beefy-R Supports Enhanced Multi-Passage Expansion of BSCs Compared with Beefy-9

Following short-term studies, multi-passage growth was assessed for BSCs in Beefy-R, Beefy-9, and a scrum-containing medium (BSC-GM) (FIG. 3). For BSC-GM, plates were coated with 0.25 μg/cm² of recombinant laminin, while for Beefy-9 and Beefy-R, plates were coated with 1.5 μg/cm² of recombinant vitronectin, as determined during the development of Beefy-9. Similarly, as determined during Beefy-9 development, albumin and RPI were withheld from the media while seeding each passage and were added only after cells had attached to the plates overnight [7]. The results showed that Beefy-R improved growth compared with Beefy-9 over four passages (FIG. 3a). Indeed, cells grown in Beefy-R achieved 11.7 population doublings over thirteen days compared with 10.6 doublings for Beefy-9. Thus, twice as many cells grew over two weeks when albumin was replaced with RPI. However, neither serum-free media promoted growth equivalent to 20% FBS (14.8 population doublings), suggesting further optimization is required.

Comparing growth rates over four passages, the average population doubling times for Beefy-R, Beefy-9, and BSC-GM were 26.6, 30.2, and 21.3 h, respectively. While all media showed an increase in doubling time with increased passage number, Beefy-R showed slower doubling rate deterioration than Beefy-9, though faster than BSC-GM. Together, these results showed that Beefy-R improved growth over Beefy-9, though not to the degree of BSC-GM. This suggests that RPI offers enhanced functionality over albumin, but that further media optimization work is still required.

### Beefy-R Maintains BSC Identity and Myogenicity During Culture

BSCs cultured in various media were subjected to a range of analyses of both proliferating cells (70% confluency) and cells differentiated in a previously described serum-free differentiation medium [50]. First, quantitative PCR (qPCR) was performed to assess expression of four genes: the satellite cell marker Paired-box 3 (Pax3), the myogenic commitment marker Myoblast Determination Protein 1 (MyoD), the early differentiation marker Myogenin, and the terminal differentiation marker Myosin Heavy Chain (MHC) (FIG. 4a). For proliferating cells, notable differences between serum-free and serum-containing conditions existed for MyoD and Myogenin. Specifically, BSC-GM cells showed a significant (˜10-fold) increase in MyoD compared with Beefy-R or Beefy-9, and a significant (˜6-fold) decrease in Myogenin.

These results suggest that BSC-GM maintained an earlier muscle phenotype than Beefy-R or Beefy-9. For differentiating cells, notable differences existed for Myogenin and MHC, where both serum-free media showed a significant (˜3.5-fold) increase in Myogenin, and Beefy-R showed a significant (˜3-fold) increase in MHC, compared with BSC-GM. These results suggest enhanced differentiation of cells from serum-free media, which is in line with their later-stage proliferative phenotype.

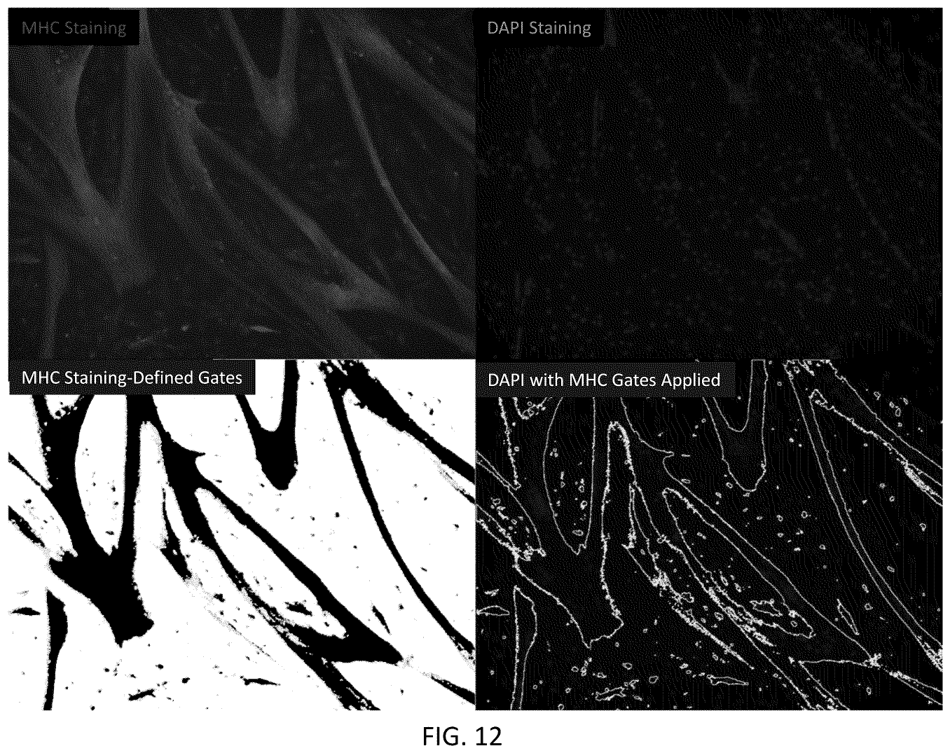

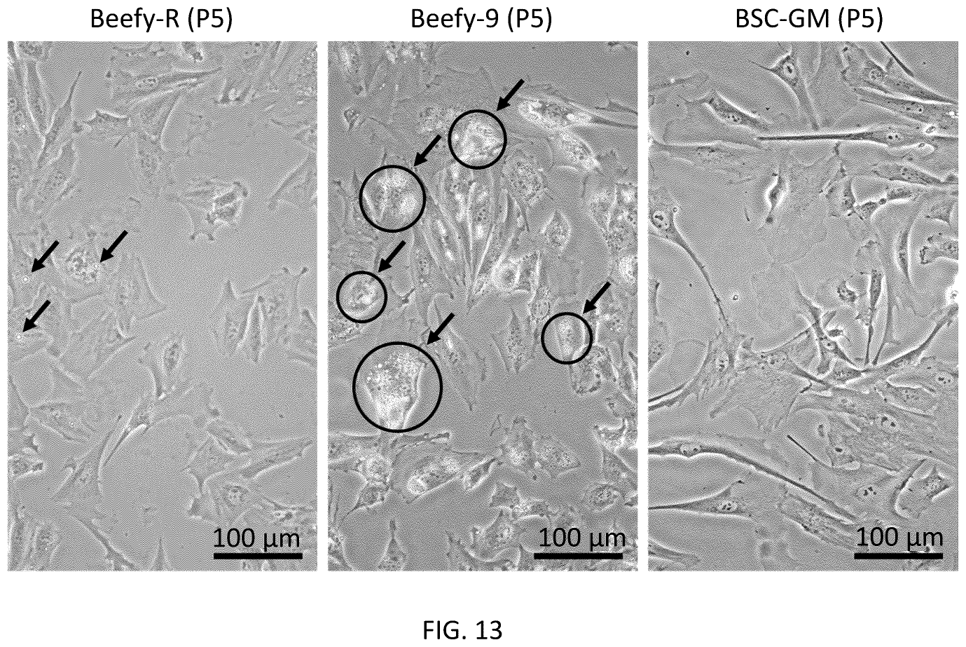

Along with qPCR, immunostaining for Pax7, MyoD, and MHC was performed on proliferative and differentiated cells. Proliferative cells showed ubiquitous expression of Pax7 and heterogeneous expression of MyoD for all media (FIGS. 4b & 9). Over 99% of cells were Pax7-positive in all three media, suggesting that all could maintain satellite cell identity to a high degree (FIGS. 4c and 10). Differentiated cells showed myotube fusion and MHC staining for all media, indicating robust differentiation (FIGS. 4d & 11). Image analysis of MHC staining was performed to assess fusion index, or the percentage of nuclei that are present in MHC-positive myotubes (FIG. 12). The results showed increased fusion index for Beefy-R and Beefy-9 compared with BSC-GM (FIG. 4c), which supports the qPCR results. Finally, brightfield images of passage 5 cells showed reduced lipid droplet formation in both Beefy-R and BSC-GM compared with Beefy-9 (FIG. 13). This is notable, as abnormal lipid accumulation was observed during the development of Beefy-9, and so Beefy-R could reduce this phenotypic aberration [7].

Together, these data revealed that RPI maintained myogenic capacity at least as well as albumin, but that both Beefy-R and Beefy-9 resulted in a later-stage phenotype than BSC-GM. This difference resulted in enhanced differentiation compared with BSC-GM, although this could also mean reduced stemness and proliferative capacity over longer-term culture. Ultimately, these results support the utility of RPI in serum-free culture and the need for further Beefy-R media optimization.

### Different Lots of Rapeseed Protein Cake Reveal Heterogeneity in RPI

Next, Beefy-R growth analysis was performed using two different lots of rapeseed protein cakes and a previously developed immortalized bovine satellite cell (iBSC) line [25]. This was done for two reasons. The first is that significant variations in the composition of rapeseed protein cakes can be seen across climates, regions, and even processing facilities [51,52]. Therefore, by comparing two different lots of protein cakes (from two different years of rapeseed harvest), some insight can be gained RPI variations from different sources. The second reason is that, as seen in FIG. 3, cell growth of primary BSCs begins to slow down at higher passages. One likely explanation for this is cell senescence; however, it is necessary to test Beefy-R with immortalized cells to verify this. Additionally, by testing Beefy-R with iBSCs, longer studies are possible which can help to highlight differences between media by compounding those differences over long-term growth.

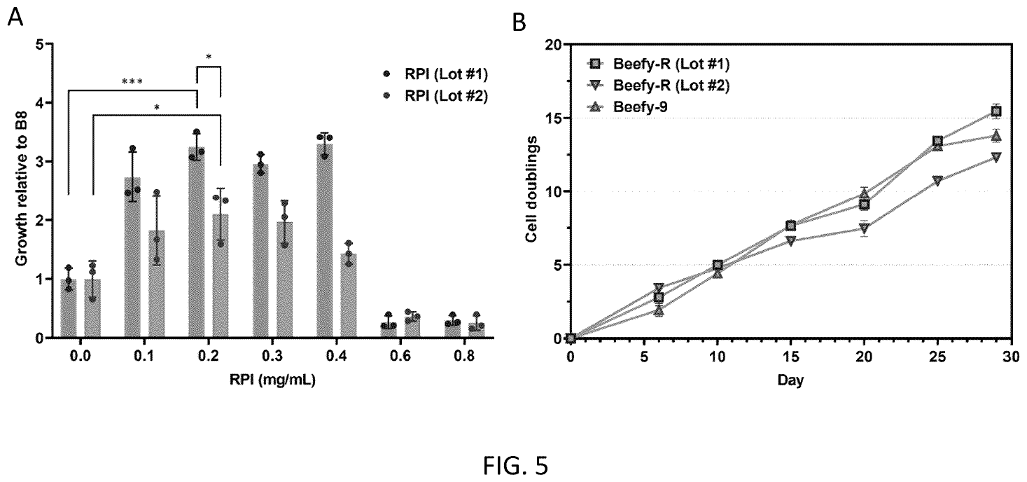

To perform this study, preliminary short-term screens were first performed as before, using newly prepared RPI from Lot #1 of rapeseed protein cakes (the same lot used in earlier parts of this study and comprised of rapeseed sourced in 2021) and RPI prepared from Lot #2 of rapeseed protein cakes (comprised of rapeseed sourced in 2022). The results showed that RPI from Lot #2 induced relatively less cell growth compared with RPI from Lot #1 (FIG. 5a). The results also showed that, while 0.4 mg/mL remained optimal for Lot #1, 0.2 mg/mL was optimal for Lot #2. Because of this, 0.2 mg/mL was used to generate Beefy-R for long term iBSC growth analysis in order to directly compare the two sources of RPI. The results from this study showed that both Lot #1 and Lot #2 of RPI promoted continuous growth over ˜ 1 month, with no clear slow-down at higher passages (FIG. 5b). Results also showed that iBSCs grew faster in Lot #1 of Beefy-R, compared with Lot #2. Specifically, cells in Lot #1 of Beefy-R grew at a comparable rate to Beefy-9, while cells in Lot #2 of Beefy-R experienced ˜3 fewer doublings over the duration of the experiment (FIG. 5). Together, these results show that Beefy-R is effective for long-term culture of immortalized bovine satellite cells, but that different lots of rapeseed protein cakes show varying efficacy. While both preparations of Beefy-R supported long-term cell growth, this variation could have substantial impacts on cultured meat production when compounded at scale. As such, future work should focus on optimizing RPI preparation to account for and minimize batch-to-batch variability between protein isolates.

### Proteomic Analysis Reveals Differences Between OPI Composition

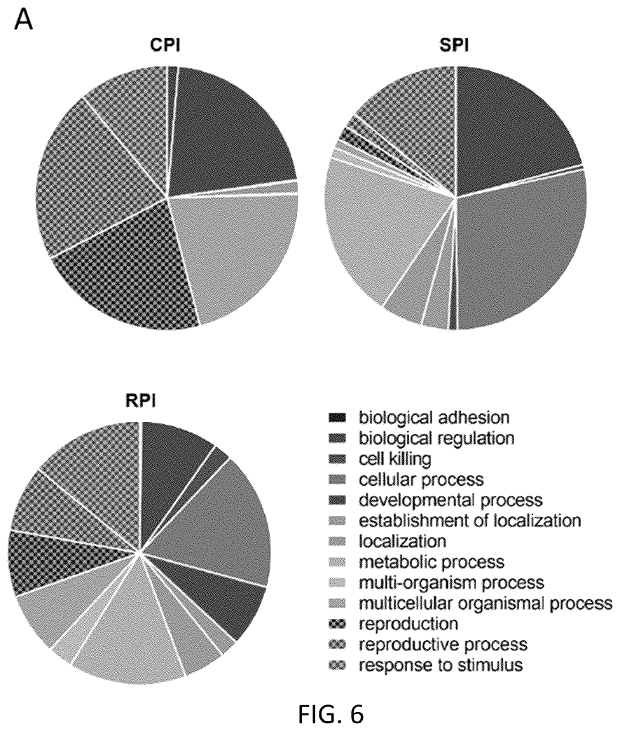

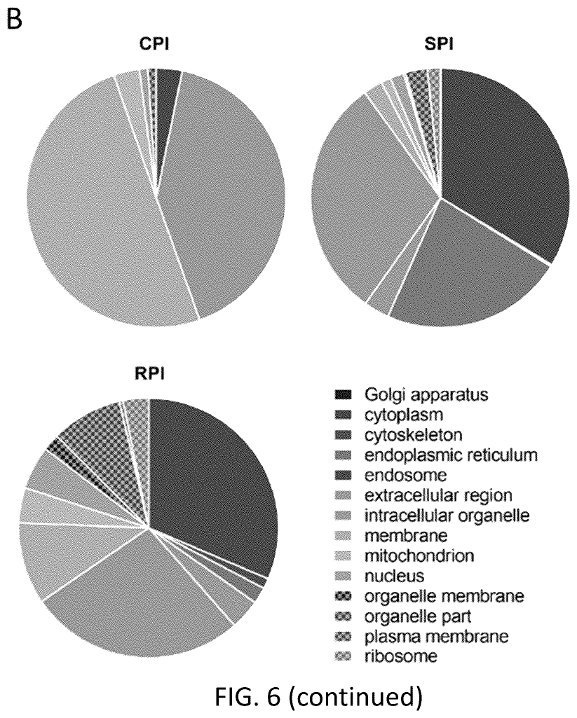

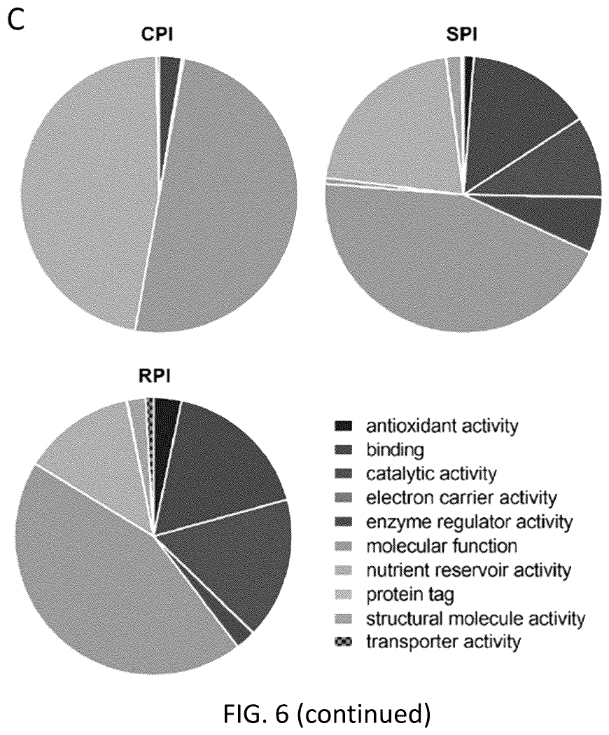

Proteomic and Gene Ontology (GO) analyses were performed on OPIs that improved growth (CPI, SPI and RPI-Lot #1). Identified proteins were weighted by Normalized Spectral Abundance Factor (NSAF) and categorized by biological process, cellular component, and molecular function. The results showed that, while all OPIs differed, SPI and RPI were more similar to each other than to CPI. For instance, looking at biological processes (FIG. 6a), both SPI and RPI had a higher prevalence of proteins involved in cellular processes and metabolic processes. Looking at cellular components (FIG. 6b), SPI and RPI had a higher prevalence of cytoplasmic proteins, while CPI had a substantially higher prevalence of membrane proteins. Finally, looking at molecular functions (FIG. 6c), SPI and RPI had a higher prevalence of binding and catalytic proteins, while CPI had a higher prevalence of nutrient reservoir proteins. It is possible that the enhanced efficacy of SPI and RPI is attributable to these trends in composition.

Looking more closely at protein composition, relative abundance of specific proteins was assessed through the calculation of Normalized Spectral Abundance Factor (NSAF), which provides a relative measure of protein abundance for a given OPI [26,27]. Results revealed the most prevalent proteins in all three OPIs (Table 2-3). For RPI, the most prevalent protein was Napin, a 2S albumin which comprised ˜6% of proteins, according to NSAF. For SPI, the most prevalent protein was trypsin inhibitor A, which comprised ˜6% of proteins, according to NSAF. For CPI, the most prevalent was Legumin A, an 11S globulin which comprised ˜20% of proteins, according to NSAF. Overall, RPI had the highest relative abundance of albumins (10.68%) with the lowest relative abundance of globulins (10.1%). This is in comparison with 4.24% albumins and 33.02% globulins for SPI, and 6.02% albumins and 53.88% globulins for CPI. These results correspond with SDS-Page results in FIG. 1, which suggested predominantly albumins and globulins for OPIs. RPI also showed a relatively high abundance of oil body-associated proteins (OBAPs) and oleosins, while SPI and CPI both showed relatively high levels of late embryogenesis abundant proteins (LEAs).

TABLE 2
Relative abundance of the most prevalent proteins in RPI, SPI and
CPI (determined from Normalized Spectral Abundance Factor, NSAF), as well as relative
abundance of total albumins and globulins in these OPIs.

| Relative protein | Rapeseed | | Soybean | | Cottonseed | |
|---|---|---|---|---|---|---|
| abundance | Category | Σ(NSAF) | Category | Σ(NSAF) | Category | Σ(NSAF) |
| 1st most abundant | 28 albumins | 10.68% | 118 globulins | 19.38% | 315 globulins | 36.65% |
| 2nd most abundant | 115 globulins | 10.10% | 75 globulins | 13.64% | 78 globulins | 17.23% |
| 3rd most abundant | Oil body-associated | 5.69% | Late embryogenesis | 4.46% | 28 albumins | 6.02% |
| | proteins | | abundant proteins | | | |
| 4th most abundant | Oleosions | 3.43% | 28 albumins | 4.24% | Late embryogenesis | 6.01% |
| | | | | | abundant | |
| | | | | | proteins | |
| Total albumins | | 10.60% | | 4.24% | | 6.02% |
| Total globulins | | 10.10% | | 33.02% | | 53.58% |
| Identified proteins | | 634 | | 389 | | 58 |

TABLE 3
Protein abundance

| | Protein family | Accession | Description | Avg NSAF | Stdv |
|---|---|---|---|---|---|
| Rapeseed | 2S seed storage albumins family | A0A078IPA5 | Napin (embryo-specific) | 0.0558 | 0.0015 |
| | OBAP family | A0A078HPD4 | Oil body-associated protein 2A | 0.0277 | 0.0005 |
| | 11S seed storage protein (globulins) family | A0A078HBQ3 | Cruciferin CRU1 | 0.0226 | 0.0008 |
| | 2S seed storage albumins family | A0A078H4D8 | Napin-2 | 0.0220 | 0.0006 |
| | 11S seed storage protein (globulins) family | A0A078G0Q2 | Cruciferin CRU4 | 0.0218 | 0.0013 |
| | OBAP family | A0A078FAS4 | Oil body-associated protein 1A | 0.0209 | 0.0007 |
| | 11S seed storage protein (globulins) family | A0A078GA98 | Cruciferin BnC1 | 0.0194 | 0.0008 |
| | Jacalin lectin family | ADA07815W6 | Jacalin-related lectin 36 | 0.0180 | 0.0011 |
| | Plant dehydrin family | A0A078IUD1 | Dehydrin | 0.0157 | 0.0029 |
| | 11S seed storage protein (globulins) family | A0A078FAN5 | 12S seed storage protein CRD | 0.0156 | 0.0010 |
| | | A0A078FWT5 | 3-oxoacyl-Jacyl-carrier-protein] reductase | 0.0135 | 0.0003 |
| | 2S seed storage albumins family | A0A078IBF3 | Napin-1A-like | 0.0130 | 0.0015 |
| | 2S seed storage albumins family | A0A078I987 | Napin-B-like | 0.0124 | 0.0002 |
| | | A0A078I535 | Kunitz trypsin inhibitor 4-like | 0.0118 | 0.0006 |
| | Jacalin lectin family | A0A078J5K9 | Jacalin-related lectin | 0.0116 | 0.0003 |
| | | | Sum of top 15 proteins (NSAF) | 0.3018 | |
| | | albumins | Total albumins (2S; all proteins) | 0.1068 | |
| | | globulins | Total 7S globulins (all proteins) | 0.0000 | |
| | | | Total 11S globulins (all proteins) | 0.1010 | |
| | | | Total globulins (7S & 11S; all proteins) | 0.1010 | |
| Soybean | Protease inhibitor I3 (leguminous Kunitz) | P01070 | Trypsin inhibitor A | 0.0597 | 0.0009 |
| | 7S seed storage protein family | P0DO16 | Beta-conglycinin alpha subunit 1 | 0.0532 | 0.0008 |
| | 7S seed storage protein family | F7J077 | Beta-conglycinin beta subunit 2 | 0.0459 | 0.0012 |
| | 11S seed storage protein (globulins) family | P04405 | Glycinin G2 | 0.0453 | 0.0008 |
| | 11S seed storage protein (globulins) family | P04776 | Glycinin G1 | 0.0433 | 0.0014 |
| | 11S seed storage protein (globulins) family | A0A0R0GMV1 | Glycinin G5 | 0.0421 | 0.0011 |
| | 11S seed storage protein (globulins) family | P02858 | Glycinin G4 | 0.0407 | 0.0004 |
| | 7S seed storage protein family | P11827 | Beta-conglycinin alpha prime subunit | 0.0374 | 0.0007 |
| | 2S seed storage albumins family | P19594 | 2S albumin | 0.0278 | 0.0002 |
| | 11S seed storage protein (globulins) family | P11828 | Glycinin G3 | 0.0224 | 0.0004 |
| | Peptidase C1 family | O64458 | P34 probable thiol protease | 0.0162 | 0.0007 |
| | Leguminous lectin family | P05046 | Lectin | 0.0160 | 0 0007 |
| | 2S seed storage albumins family | Q9ZNZ4 | Napin-type 2S albumin 1 precursor | 0.0146 | 0.0012 |
| | Small heat shock protein (HSP20) family | Q9XET1 | Seed maturation protein PM31 | 0.0145 | 0 0007 |
| | Lipoxygenase family | B3TDK6 | Lipoxygenase-3 | 0.0145 | 0.0001 |
| | | | Sum of top 15 proteins (NSAF) | 0.4935 | |
| | | albumins | Total albumins (2S; all proteins) | 0.0424 | |
| | | globulins | Total 7S globulins (all proteins) | 0.1364 | |
| | | | Total 11S globulins (all proteins) | 0.1938 | |
| | | | Total globulins (7S & 11S; all proteins) | 0.3302 | |
| Cottonseed | 11S seed storage protein (globulins) family | A0A1U8KKK7 | Legumin A-like | 0.2041 | 0.0040 |
| | 7S seed storage protein family | A0A1U8LMX7 | Vicilin GC72-A | 0.1723 | 0.0026 |
| | 11S seed storage protein (globulins) family | A0A1USKAE1 | Lecumin B-like | 0.1623 | 0.0062 |
| | Small hydrophilic plant seed protein family | Q53WZ7 | Late embryogenesis abundant protein D-19 | 0.0910 | 0.0047 |
| | | Q03791 | Embryogenesis abundant proteins | 0.0478 | 0.0001 |
| | | A0A1U8LZX8 | Violin Pis v 3.0101-like | 0.0376 | 0.0022 |
| | 2S seed storage albumins family | A0A1U8PKJ6 | 2S albumin seed storage protein-like | 0.0369 | 0.0021 |
| | Small hydrophilic plant seed protein family | A0A1U8NGV9 | Em protein H5-like | 0.0292 | 0.0061 |
| | LEA type 1 family | A0A1U8HTY4 | Late embryogenesis abundant protein D- | 0.0288 | 0.0030 |
| | | | 113 | | |
| | 2S seed storage albumins family | A0A1U8LLD2 | 2S sulfur-rich seed storage protein 1-like | 0.0233 | 0.0013 |
| | Plant dehydrin family | A0A1U8ILV7 | Late embryogenesis abundant protein D-11 | 10.0186 | 0.0013 |
| | | A0A1U8K9G0 | Uncharacterized protein LOC107913263 | 0.0184 | 0.0034 |
| | LEA type SMP family | A0A1U8LI10 | Late embryogenesis abundant protein D-34 | 0.0176 | 0.0010 |
| | Oleosin family | A0A1U8LH53 | Oleosin | 0.0170 | 0.0004 |
| | | A0A1U8I8C9 | late embryogenesis abundant protein D-7 | 0.0143 | 0.0012 |
| | | | Sum of top 15 proteins (NSAF) | 0.9194 | |
| | | | Total albumins (2S; all proteins) | 0.0602 | |
| | | | Total 7S globulins (all proteins) | 0.1723 | |
| | | | Total 11S globulins (all proteins) | 0.3665 | |
| | | | Total globulins (7S & 11S; all proteins) | 0.5388 | |

The increased concentration of 2S albumin could have had some impact on the increased efficacy of RPI as a serum albumin replacement in Beefy-R, though further research is required to parse the contributions of specific proteins. Specifically, protein fractionation would serve as a valuable next step for assessing the functional effect of certain proteins, and for optimizing extraction methods to maximize OPI efficacy. This would also help to elucidate the impact of different concentrations of specific protein components on cells. For instance, NSAF data for RPI suggests that Beefy-R contains ˜0.04 mg/mL of 2S albumins. This is in contrast with Beefy-9 or BSC-GM, which contain 0.8 mg/mL of recombinant human serum albumin or ˜5 mg/mL of bovine serum albumin, respectively [53]. Understanding the relative functional contributions of specific proteins (both beneficial and growth-inhibiting) and tuning levels of those proteins could substantially improve Beefy-R outcomes. This is particularly true given that 2S albumins are less similar to serum albumins than their name might imply. Indeed, while serum albumins function to bind ligands, fatty acids, ions, and other factors, and to maintain blood osmolarity [54,55], 2S albumins act primarily as nutrient storage reservoirs for germinating seeds, with some concurrent antimicrobial activity [56]. As such, it might be other proteins (or combinations of proteins) which act to replace albumin function in Beefy-R, rather than just the effect of the highly prevalent 2S albumins or globulins. For instance, oil-body associated proteins (OBAPS) are most prevalent in RPI and could potentially offer functionality similar to serum albumin's fatty acid binding role [57]. Again, further fractionation and study of specific proteins is needed to determine which OPI proteins offer which functions, and to maximize the extraction and utilization of those functional proteins. Finally, while NSAF is considered a suitable metric for analyzing relative protein abundance, further quantitative assessments should be performed to better understand precise protein composition of OPIs and help elucidate functional differences.

### CONCLUSIONS

Replacing recombinant albumin in serum-free media is a key priority for cultured meat research. Specifically, due to the high concentrations of albumin required in relevant serum-free media to-date, it is expected that recombinant techniques-even at their most efficient-will be unable to provide sufficiently affordable protein for low-cost bioprocesses [58]. It has therefore been suggested that albumin alternatives are necessary to achieve economic viability at scale. These alternatives should be effective, low-cost, scalable, food-safe, and sustainable. Agricultural byproducts offer many advantages towards achieving these goals, including the valorization of high-volume waste streams and availability of low-cost inputs which can be processed in a simple, food safe manner. In this work, an effective serum-free medium (termed Beefy-R) was developed as validation of the efficacy of non-hydrolyzed oilseed protein isolates (particularly rapeseed protein isolate) as effective albumin alternatives. RPI is easy to produce through simple and low-cost steps and was also shown to exceed albumin in promoting BSC growth while maintaining myogenicity.

While determining total cost-of-goods of RPI at scale (including processing such as electricity, filtration, chemicals, and waste management) is outside the scope of this work, the low cost of input materials and simplicity/scalability of extraction and processing should result in a highly affordable media component. Specifically, the cost of input material (rapeseed protein cakes) required to make enough RPI for 1 L of Beefy-R at 0.4 mg/mL of RPI is $0.002. The real-world cost contribution will be higher in practice, once processing costs are factored in; however, they will likely still be orders of magnitude lower than recombinant albumin's cost contribution to Beefy-9, which is approximately $24.56 per liter when purchasing from available suppliers [7].

After overcoming the high cost of recombinant albumin with RPI, FGF-2 remains a key cost contributor in Beefy-R. Here, price is driven by the currently high cost of FGF-2, rather than the concentration used (40 ng/mL). As such, it is likely that process scale-up can drive down costs substantially. Methods to optimize and enhance growth factors or overcome their requirement in cell culture could also reduce these costs [8,59-62]. Additionally, during Beefy-9 development, it was shown that lowering the concentration of FGF-2 to 5 ng/mL did not drastically reduce growth, and so this simple strategy could reduce the cost of Beefy-R further. Similarly, recent studies have shown robust BSC growth in serum-free media containing reduced insulin (50% that of Beefy-9), reduced FGF-2 (25% that of Beefy-9), and reduced transferrin (27.5% that of Beefy-9) [63]. Thus, reducing these components could be explored to optimize Beefy-R. Finally, it should be noted that the protein isolation process used in this work leaves behind a number of proteins that are insoluble at physiological pH, and which could themselves be useful byproducts for the production of resources such as scaffolds. For instance, soy protein isolates have been used to form various scaffolds for culturing BSCs for cultured meat [64,65]. It is possible that similar scaffolds could be generated using the rapeseed protein fraction which remains after the steps used in this study, thereby reducing the cost of RPI by valorizing the unused portion.

From a consumer and regulatory standpoint, several features of RPI should be considered and studied prior to implementation in cultured meat development. Specifically, the food-safety of RPI must be validated as appropriate for cultured meat production [66]. Indeed, rapeseed protein meal is typically used as animal feed or fertilizer, and so does not have the same quality control requirements that should be established for cultured meat. Possible hazards include contaminating microorganisms that must be removed to use RPI for culture media. In this study, filtration was used to sterilize RPI; however, other methods might be required at scale. Additionally, oilseed proteins can be allergenic for some consumers, including 2S albumins and 11S globulins prevalent in OPIs [67]. Further characterization should therefore be performed to determine the allergenicity of OPIs, and whether or not any allergens are incorporated into a final meat product that uses cells which were expanded with Beefy-R. Finally, the sustainability and scalability of RPI and other OPIs should be validated through dedicated techno-economic and life-cycle analyses, in order to ensure that this technology contributes positively to cultured meat's impact on our food system [75].

Further, while this work confirms the utility of RPI as an albumin alternative, substantial future work should be pursued to further explore OPIs and other plant protein extracts. This could include fractionating OPIs to identify functional constituents, optimizing methods to maximize yields and efficacy, establishing methods to minimize batch-to-batch variability, exploring other oilseeds and agricultural waste streams for functional components, and optimizing other media com-ponents for enhanced growth and differentiation [63,68]. Additionally, future work could explore plant protein extracts as alternatives for other costly recombinant proteins, such as insulin and transferrin, and for other cell types, such as preadipocytes and iPSCs. Here, computational methods can be leveraged to identify additional low-cost inputs (e.g., functional protein fractions) and optimize media formulations [69,70]. Finally, when considering RPI's demonstrated efficacy and potential future work, it is important to note the substantial body of work that exists exploring albumin alternatives for various applications. In this study, a number of these other alternatives were explored, including cyclodextrins (which have been demonstrated as effective albumin alternatives for lymphocytes) [47], hydrolysates from soy, wheat, and yeast (which have been demonstrated as albumin alternatives for kidney cell culture, albumin alternatives for in vitro embryo maintenance, and serum-free media additives for CHO cells, respectively) [12, 48, 71], and molecular crowding agents (dextran and polyethylene glycol), as crowding is thought to be a potential source of the beneficial effects of albumin at high concentrations [49]. While none of these additives effectively substituted albumin in Beefy-9 for BSCs, it is possible that combinations of OPIs with these other factors could have beneficial effects on cell culture outcomes. Similarly, other albumin alternatives could be explored in conjunction with Beefy-R, including rapeseed protein hydrolysates, pea and chickpea albumin analogues, emulsifiers such as methylcellulose, or fatty acid-binding proteins such as beta-lactoglobulin, all of which have been explored previously as albumin alternatives in cell culture applications [10,72-74].

In summary, the present work validates the utility of OPIs, particularly RPI, in replacing albumin in serum-free media for BSCs. The resulting media, Beefy-R, enhanced cell growth compared with albumin-containing Beefy-9 while reducing costs and maintaining satellite cell phenotype and myogenicity. Protein analysis of OPIs revealed compositional differences which likely correlate with functional disparities. While substantial opportunities for further research and optimization exist, this work addresses a key challenge facing cultured meat development and lays a foundation for achieving cost-effective serum-free media formulations.

### REFERENCES

1. H. C. J. Godfray, et al., Meat consumption, health, and the environment, Science 361 (2018), eaam5324.
2. C. S. Mattick, A. E. Landis, B. R. Allenby, N. J. Genovese, Anticipatory life cycle analysis of in vitro biomass cultivation for cultured meat production in the United States, Environ. Sci. Technol. 49 (2015) 11941-11949.
3. M. J. Post, et al., Scientific, sustainability and regulatory challenges of cultured meat, Nat. Food 1 (2020) 403-415.
4. H. L. Tuomisto, S. J. Allan, M. J. Ellis, Prospective life cycle assessment of a bioprocess design for cultured meat production in hollow fiber bioreactors, Sci. Total Environ. 851 (2022), 158051.
5. D. Humbird, Scale-up economics for cultured meat, Biotechnol. Bioeng. 118 (2021) 3239-3250.
6. P. G. Negulescu, et al., Techno-Economic Modelling and Assessment of Cultivated Meat: Impact of Production Bioreactor Scale, 2022, https://doi.org/10.31224/2414. Preprint at.
7. A. J. Stout, et al., Simple and effective serum-free medium for sustained expansion of bovine satellite cells for cell cultured meat, Commun. Biol. 5 (2022) 1-13.
8. H.-H. Kuo, et al., Negligible-cost and weekend-free chemically defined human iPSC culture, Stem Cell Rep. 14 (2020) 256-270.
9. TEA of cultivated meat. Future Projections for Different Scenarios. CE Delft-EN https://cedelft.cu/publications/tea-of-cultivated-meat/.
10. B. Farges-Haddani, et al., Peptide fractions of rapeseed hydrolysates as an alternative to animal proteins in CHO cell culture media, Process Biochem. 41 (2006) 2297-2304.
11. G. Chabanon, et al., Influence of the rapeseed protein hydrolysis process on CHO cell growth, Bioresour. Technol. 99 (2008) 7143-7151.
12. Y. H. Sung, S. W. Lim, J. Y. Chung, G. M. Lee, Yeast hydrolysate as a low-cost additive to serum-free medium for the production of human thrombopoietin in suspension cultures of Chinese hamster ovary cells, Appl. Microbiol. Biotechnol. 63 (2004) 527-536.
13. J. Giro\acute{ } n-Calle, et al., Chickpea protein hydrolysate as a substitute for serum in cell culture, Cytotechnology 57 (2008) 263-272.
14. F. Fran ̌ ek, O. Hohenwarter, H. Katinger, Plant protein hydrolysates:
   preparation of defined peptide fractions promoting growth and production in animal cells cultures, Biotechnol. Prog. 16 (2000) 688-692.
15. R. Christel Andreassen, E. Pedersen, M., A. Kristoffersen, K., S. B. Rønning, Screening of by-products from the food industry as growth promoting agents in serum-free media for skeletal muscle cell culture, Food Funct. 11 (2020) 2477-2488.
16. J. Pedroche, et al., Brassica carinata protein isolates: chemical composition, protein characterization and improvement of functional properties by protein hydrolysis, Food Chem. 88 (2004) 337-346.
17. S. Rawdkuen, N. Rodzi, S. Pinijsuwan, Characterization of sacha inchi protein hydrolysates produced by crude papain and Calotropis proteases, LWT 98 (2018) 18-24.
18. A. Tapal, P. K. Tiku, Complexation of curcumin with soy protein isolate and its implications on solubility and stability of curcumin, Food Chem. 130 (2012) 960-965.
19. M. Ma, et al., Physicochemical and functional properties of protein isolate obtained from cottonseed meal, Food Chem. 240 (2018) 856-862.
20. Y. Li, et al., Modification of rapeseed protein by ultrasound-assisted pH shift treatment: ultrasonic mode and frequency screening, changes in protein solubility and structural characteristics, Ultrason. Sonochem. 69 (2020), 105240.
21. B. Zhang, Y. Cui, G. Yin, X. Li, X. Zhou, Alkaline extraction method of cottonseed protein isolate, Mod. Appl. Sci. 3 (2009) p 77.
22. G. Chabanon, I. Chevalot, X. Framboisier, S. Chenu, I. Marc, Hydrolysis of rapeseed protein isolates: kinetics, characterization and functional properties of hydrolysates, Process Biochem. 42 (2007) 1419-1428.
23. S. K. Sathe, H. H. Kshirsagar, G. M. Solubilization Sharma, Fractionation, and electrophoretic characterization of inca peanut (plukenetia volubilis L.) proteins, Plant Foods Hum. Nutr. 67 (2012) 247-255.
24. C. W. Mcaleer, J. W. Rumsey, M. Stancescu, J. J. Hickman, Functional myotube formation from adult rat satellite cells in a defined serum-free system, Biotechnol. Prog. 31 (2015) 997-1003.
25. A. J. Stout, et al., Immortalized Bovine Satellite Cells for Cultured Meat Applications, 2022, https://doi.org/10.1101/2022.12.02.518927. Preprint at.
26. S. McIlwain, et al., Estimating relative abundances of proteins from shotgun proteomics data, BMC Bioinf. 13 (2012) 308.
27. A. C. Paoletti, et al., Quantitative proteomic analysis of distinct mammalian Mediator complexes using normalized spectral abundance factors, Proc. Natl. Acad. Sci. USA 103 (2006) 18928-18933.
28. Y. Perez-Riverol, et al., The PRIDE database resources in 2022: a hub for mass spectrometry-based proteomics evidences, Nucleic Acids Res. 50 (2022) D543-D552.
29. Fetzer, K. Müller, M. Schmid, P. Eisner, Rapeseed proteins for technical applications: processing, isolation, modification and functional properties—a review, Ind. Crop. Prod. 158 (2020), 112986.
30. M. Shemer, S. Mizrahi, Z. Berk, S. Mokady, Effect of processing conditions on isolation of cottonseed protein by sodium hexametaphosphate extraction method, J. Agric. Food Chem. 21 (1973) 460-462.
31. E. W. Lusas, M. N. Riaz, Soy protein products: processing and use, J. Nutr. 125 (1995) 573S-580S.
32. H. Kalaydzhiev, et al., Enhanced solubility of rapeseed meal protein isolates prepared by sequential isoelectric precipitation, Foods 9 (2020) 703.
33. Fetzer, T. Herfellner, P. Eisner, Rapeseed protein concentrates for non-food applications prepared from pre-pressed and cold-pressed press cake via acidic precipitation and ultrafiltration, Ind. Crop. Prod. 132 (2019) 396-406.
34. D. Verfaillie, F. Janssen, G. Van Royen, A. G. B. Wouters, A systematic study of the impact of the isoelectric precipitation process on the physical properties and protein composition of soy protein isolates, Food Res. Int. 163 (2023), 112177.
35. Y. Zhao, R. Tian, Z. Xu, L. Jiang, X. Sui, Recent advances in soy protein extraction technology, J. Am. Oil Chem. Soc. 1 (2022) 9, https://doi.org/10.1002/aocs.12676.
36. Foreign Agriculture Service, (USDA), World Markets and Trade, Oilseeds, 2022.
37. P. Li, et al., Structural, functional properties and immunomodulatory activity of isolated Inca peanut (Plukenetia volubilis L.) seed albumin fraction, Int. J. Biol. Macromol. 118 (2018) 1931-1941.
38. E. Ntone, et al., Napins and cruciferins in rapeseed protein extracts have complementary roles in structuring emulsion-filled gels, Food Hydrocolloids 125 (2022), 107400.
39. N. Chen, M. Zhao, C. Chassenieux, T. Nicolai, Data on the characterization of native soy globulin by SDS-Page, light scattering and titration, Data Brief 9 (2016) 749-752.
40. K. Gandhi, N. Litoriya, A. Shah, J. G. Talati, Extraction, fractionation and characterization of cotton (Gossypium herbaceum L.) seed proteins, Indian J. Agric. Biochem. 30 (2017) 21-26.
41. Z. He, D. Zhang, H. Cao, Protein profiling of water and alkali soluble cottonseed protein isolates, Sci. Rep. 8 (2018) 9306.
42. L. Day, Protein: food sources, in: B. Caballero, P. M. Finglas, F. Toldra\acute{ } (Eds.), Encyclopedia of Food and Health, Academic Press, 2016, pp. 530-537, https://doi. org/10.1016/B978-O-12-384947-2.00576-6.
43. A. A. Sadeghi, P. Shawrang, Effects of microwave irradiation on ruminal protein degradation and intestinal digestibility of cottonseed meal, Livest. Sci. 106 (2007) 176-181.
44. A. L. Bittencourt, et al., Immunogenicity and allergenicity of 2S, 7S and 11S soy protein fractions, Rev. Bras. Ciencias Farm. 43 (2007) 597-606.
45. A. J. Stout, A. B. Mirliani, E. L. Soule-Albridge, J. M. Cohen, D. L. Kaplan, Engineering carotenoid production in mammalian cells for nutritionally enhanced cell-cultured foods, Metab. Eng. 62 (2020) 126-137.
46. Yamane, M. Kan, H. Hoshi, Y. Minamoto, Primary culture of human diploid cells and its long-term transfer in a serum-free medium, Exp. Cell Res. 134 (1981) 470-474.
47. Yamane, M. Kan, Y. Minamoto, Y. Amatsuji, α-Cyclodextrin, a novel substitute for bovine albumin in serum-free culture of mammalian cells, Proc. Jpn. Acad. Ser. B 57 (1981) 385-389.
48. F. George, et al., Plant protein hydrolysates (plant peptones) as substitutes for animal proteins in embryo culture medium, Reprod. Fertil. Dev. 21 (2009) 587-598.
49. D. Tsiapalis, D. I. Zeugolis, It is time to crowd your cell culture media-physicochemical considerations with biological consequences, Biomaterials 275 (2021), 120943.
50. C. W. McAleer, J. W. Rumscy, M. Stancescu, J. J. Hickman, Functional myotube formation from adult rat satellite cells in a defined serum-free system, Biotechnol. Prog. 31 (2015) 997-1003.
51. J. M. Bell, M. O. Keith, A survey of variation in the chemical composition of commercial canola meal produced in Western Canadian crushing plants, Can. J. Anim. Sci. 71 (1991) 469-480.
52. P. Si, R. J. Mailer, N. Galwey, D. W. Turner, Influence of genotype and environment on oil and protein concentrations of canola (Brassica napus L.) grown across southern Australia, Aust. J. Agric. Res. 54 (2003) 397-407.
53. X. Zheng, et al., Proteomic analysis for the assessment of different lots of fetal bovine serum as a raw material for cell culture. Part IV. Application of proteomics to the manufacture of biological drugs, Biotechnol. Prog. 22 (2006) 1294-1300.
54. G. J. Quinlan, G. S. Martin, T. W. Evans, Albumin: biochemical properties and therapeutic potential, Hepatology 41 (2005) 1211.
55. V. Mishra, R. J. Heath, Structural and biochemical features of human serum albumin essential for eukaryotic cell culture, Int. J. Mol. Sci. 22 (2021) 8411.
56. P. R. Shewry, Plant storage proteins, Biol. Rev. 70 (1995) 375-426.
57. Q. Shao, X. Liu, T. Su, C. Ma, P. Wang, New insights into the role of seed oil body proteins in metabolism and plant development, Front. Plant Sci. 10 (2019) 1568.
58. S. Hubalek, M. J. Post, P. Moutsatsou, Towards resource- and cost-efficient cultured meat, Curr. Opin. Food Sci. 100885 (2022), https://doi.org/10.1016/j. cofs.2022.100885.
59. D. Nawrocka, M. A. Krzyscik, Ł. Opalin′ski, M. Zakrzewska, J. Otlewski, Stable fibroblast growth factor 2 dimers with high pro-survival and mitogenic potential, Int. J. Mol. Sci. 21 (2020) 4108.
60. X. Ren, M. Zhao, B. Lash, M. M. Martino, Z. Julier, Growth factor engineering strategies for regenerative medicine applications, Front. Bioeng. Biotechnol. 7 (2020).
61. S. C. O. Pak, S. M. N. Hunt, M. W. Bridges, M. J. Sleigh, P. P. Gray, Super-CHO a cell line capable of autocrine growth under fully defined protein-free conditions, Cytotechnology 22 (1996) 139-146.
62. M. Venkatesan, et al., Recombinant production of growth factors for application in cell culture, iScience 25 (2022).
63. Kolkmann, A. Essen, M. Post, P. Moutsatsou, Development of a chemically defined medium for in vitro expansion of primary bovine satellite cells, Front. Bioeng. Biotechnol. 10 (2022).
64. Ianovici, Y. Zagury, I. Redenski, N. Lavon, S. Levenberg, 3D-printable plant protein-enriched scaffolds for cultivated meat development, Biomaterials 284 (2022), 121487.
65. N. Xiang, et al., Edible films for cultivated meat production, Biomaterials 287 (2022), 121659.
66. K. J. Ong, et al., Food safety considerations and research priorities for the cultured meat and seafood industry, Compr. Rev. Food Sci. Food Saf. 20 (2021) 5421-5448.
67. H. Breiteneder, E. N. C. Mills, Plant food allergens-structural and functional aspects of allergenicity, Biotechnol. Adv. 23 (2005) 395-399.
68. E. N. O'Neill, Z. A. Cosenza, K. Baar, D. E. Block, Considerations for the development of cost-effective cell culture media for cultivated meat production, Compr. Rev. Food Sci. Food Saf. 20 (2021) 686-709.
69. Z. Cosenza, R. Astudillo, P. Frazier, I., K. Baar, D. E. Block, Multi-information source Bayesian optimization of culture media for cellular agriculture, Biotechnol. Bioeng. 1-12 (2022), https://doi.org/10.1002/bit.28132.
70. V. Gligorijevi\acute{ } c, et al., Structure-based protein function prediction using graph convolutional networks, Nat. Commun. 12 (2021) 3168.
71. D. W. Jayme, S. R. Smith, Media formulation options and manufacturing process controls to safeguard against introduction of animal origin contaminants in animal cell culture, Cytotechnology 33 (2000) 27-36.
72. K. Nilausen, Role of fatty acids in growth-promoting effect of serum albumin on hamster cells in vitro, J. Cell. Physiol. 96 (1978) 1-13.
73. Y. Nahmias, M. Cohen, J. Caspi, Utilization of Plant Protein Homologues in Culture Media, 2021.
74. L. Schenzle, K. Egger, A. Fuchs, H. Pichler, Never Let Me Down: Optimizing Performance of Serum Free Culture Medium for Bovine Satellite Cells, 2022, https://doi.org/10.1101/2022.11.13.516330. Preprint at.
75. D. Holmes, D. Humbird, J. Dutkiewicz, et al., Cultured meat needs a race to mission not a race to market, Nat. Food. 3 (2022) 785-787, https://doi.org/10.1038/s43016-022-00586-9.

### Example 3: Plant Proteins in Media Development for Mackerel Cells

The inventors tested several candidates of protein sources that have been reported to contain high content of soluble proteins (albumins) as reported by Loveday (2019) using dsDNA quantification. The testing process proceeded as follows: First, test each protein isolate one by one, and second, combine the protein isolate with other components (e.g., insulin, selenium, growth factors, lipids, etc.) to see if the isolate can replace with albumins.

Here, the inventors tested the ability of rapeseed protein isolate (RPI) to replace fetal bovine serum (FBS) in media for growing Mack and low serum (LS) adapted Mack1 cells.

### Experiment Setup

### Routine Cell Culture

Cells were seeded in Leibovitz's L-15 medium (11415064, Thermo Fisher Scientific, Waltham, MA, USA) with 20% fetal bovine serum (FBS) (26140079, Thermo Fisher Scientific), 20 mM HEPES (H4034, Sigma Aldrich, St. Louis, MO, USA) buffer solution (pH 7.4), 1% Antibiotic-Antimycotic (1540062, Thermo Fisher Scientific), 1 ng/mL recombinant human fibroblast growth factor (FGF-basic 154 a.a., 100-18B, PeproTech, Cranbury, NJ, USA), and 10 μg/mL gentamicin (G1397, Sigma Aldrich). The cells were incubated at 27° C. without CO₂ and detached for subculture using 0.05% trypsin-EDTA (25300054, Thermo Fisher Scientific) when they reached 70˜80% confluency.

### Experimental Design

Mack1 (available through Kerafast, #ETU008-FP) or low serum (LS) adapted Mack1 (cultured in 7.5% FBS containing media over multiple passages) cells were seeded at 2,500/cm² in the growth media (either 20 or 7.5% FBS, respectively) and incubated for 3 h to let them adhere. The old media was replaced with serum-free media (that contain every component in the growth media except FBS) supplemented with protein isolates. After 3 d incubation, every well was washed once with PBS, aspirated, and stored at −80° C. for dsDNA quantification via fluorescence (i.e., fluorescence as a proxy for dsDNA quantification as a proxy for cell proliferation as a proxy for cell quantification). Quantification was performed using CyQUANT cell proliferation assay (C7026, Thermo Fisher Scientific) according to the manufacturer's instructions.

### Statistical Analysis

One-way ANOVA with Bonferroni's multiple comparisons test (adjusted α=0.05) was performed using Prism 9.0 software (GraphPad, San Diego, CA, USA). P<0.05 were treated as statistically significant, and all errors are given as means±standard deviation (n=3).

### Results

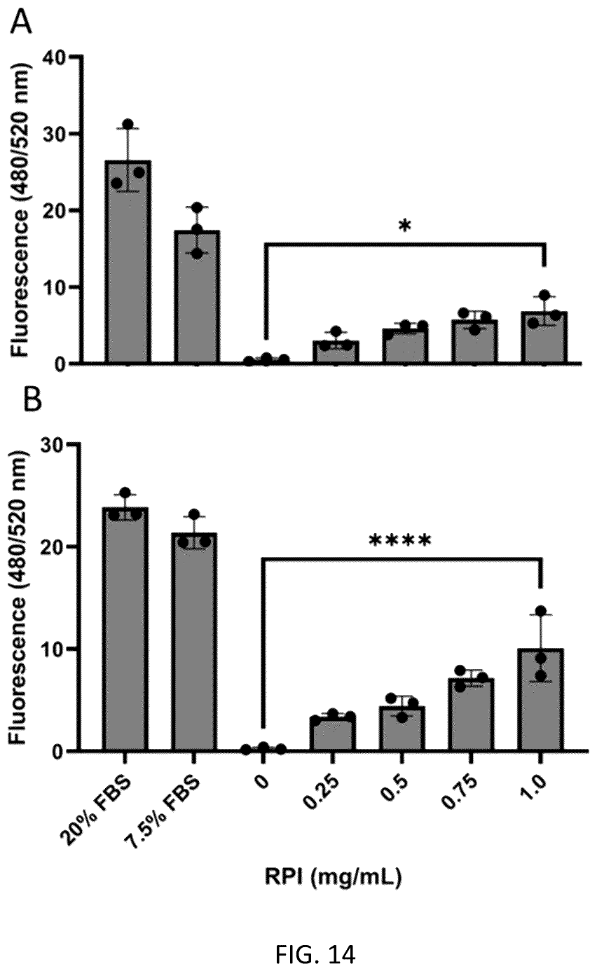

We show here that RPI can be a sufficient substitute for FBS in both Mack 1 (FIG. 14A) and LS adapted Mack1 (FIG. 14B) cells. After 3 days of incubation in FBS or RPI-supplemented media, cell viability/proliferation recovered for both types of cells when supplemented with 1.0 mg/mL RPI-supplemented media.

### REFERENCES

Loveday S M. Food Proteins: Technological, Nutritional, and Sustainability Attributes of Traditional and Emerging Proteins. Annu Rev Food Sci Technol. 2019 Mar. 25; 10:311-339. doi: 10.1146/annurev-food-032818-121128. Epub 2019 Jan. 16. PMID: 30649962.

1. A serum-free cell culture media for expansion of cells, the media comprising:
a basal culture media; and
a plant protein composition at a final concentration of 0.05 g/L to 50 g/L in the serum-free cell culture media,
wherein the plant protein composition comprises unhydrolyzed plant proteins, and wherein the serum-free cell culture media is free or substantially free of albumin.

2-3. (canceled)

4. The serum-free cell culture media of claim 1, wherein at least about 70% or 75% of the plant proteins are 10 kDa or greater in size.

5. The serum-free cell culture media of claim 1, wherein the plant protein composition comprises protein from a plant selected from the group consisting of oilseed crops, non-oilseed oil crops, legumes, pulses, and combinations thereof.

6. The serum-free cell culture media of claim 5, wherein the plant protein composition comprises protein from an oilseed crop.

7. The serum-free cell culture media of claim 1, wherein the plant protein composition comprises protein from rapeseed, soybean, sunflower seed, sesame seed, flax seed, camelina, safflower, linseed, grapeseed, pumpkin seed, poppyseed, watermelon seed, and combinations thereof.

8. The serum-free cell culture media of claim 1, wherein the plant protein composition comprises protein from rapeseed.

9-11. (canceled)

12. The serum-free cell culture media of claim 1, wherein the basal culture media comprises:
one or more sugars at a concentration of between 0.01 g/L and 10 g/L;
one or more amino acids at a concentration of between 0.001 g/L and 5 g/L;
one or more vitamins at a concentration of between 0.001 g/L and 1 g/L;
one or more trace elements at a concentration of between 0.0001 mg/L and 10 mg/L;
one or more growth-stimulating and/or cell-signaling factors at a concentration of between 0 ng/ml and 100,000 ng/ml; and
one or more transport proteins at a concentration of between 0 g/L and 1 g/L.

13. The serum-free cell culture media of claim 1, further comprising one or more of the following:
recombinant interleukin 6;
ethanolamine;
curcumin;
oleic acid;
minerals;
hepatocyte growth factor (HGF);
insulin-like growth factor 1 (IGF-1);
platelet-derived growth factor BB (PDGF-BB); and/or
linoleic acid.

14. (canceled)

15. The serum-free cell culture media of claim 1, wherein the plant protein composition is present at a concentration of at least 50 mg/L, at least 100 mg/L, at least 200 mg/L, at least 250 mg/L, at least 500 mg/L, at least 750 mg/L, at least 1 g/L, or at most 50 g/L, at most 25 g/L, at most 15 g/L, at most 10 g/L, at most 5 g/L, at most 2 g/L, at most 1.5 g/L, and at most 1 g/L or any range between these concentrations.

16. The serum-free cell culture media of claim 1, wherein the plant protein composition is present at a concentration of about 200 mg/L to about 400 mg/L.

17-22. (canceled)

23. The serum-free cell culture media of claim 1, wherein the basal culture media is selected from the group comprising B8 media, Dulbecco's Modified Eagle Medium (DMEM), Ham's F12 media, Roswell Park Memorial Institute (RPMI) 1640 media, Leibovitz's L15 and a mixture thereof.

24-29. (canceled)

30. A method of making a serum-free cell culture media, the method comprising adding a plant protein composition at a final concentration of 0.05 g/L to 50 g/L in the serum-free cell culture media to a basal culture media, thereby producing the serum-free cell culture media.

31-33. (canceled)

34. A method of expanding the numbers of or differentiating cells, the method comprising: adding cells to a serum-free cell culture media, incubating the cells in the serum-free cell culture media to allow expansion of the number of or differentiation of cells and production of growth media; and harvesting the cells and/or growth media, wherein the serum-free cell culture media comprises:
a basal culture media; and
a plant protein composition at a final concentration of 0.05 g/L to 50 g/L in the serum-free cell culture media,
wherein the plant protein composition comprises unhydrolyzed plant proteins, and
wherein the serum-free cell culture media is free or substantially free of albumin.

35. (canceled)

36. The method of claim 34, wherein at least about 74% of the plant proteins are 10 kDa or greater in size.

37. The method of claim 34, wherein the cells are selected from the group consisting of bovine, galline, ovine, porcine, equine, murine, caprine, lapine, and piscine.

38-54. (canceled)

55. The method of claim 34, wherein the plant protein composition is from a plant selected from the group consisting of oilseed crops, non-oilseed oil crops, legumes, pulses, and combinations thereof.

56. (canceled)

57. The method of claim 34, wherein the plant protein composition is from rapeseed, soybean, sunflower seed, sesame seed, flax seed, camelina, safflower, linseed, grapeseed, pumpkin seed, poppyseed, watermelon seed, and combinations thereof.

58. The method of claim 34, wherein the plant protein composition is from rapeseed.

59-61. (canceled)

62. The method of claim 34, wherein the basal culture media comprises:
one or more sugars at a concentration of between 0.01 g/L and 10 g/L;
one or more amino acids at a concentration of between 0.001 g/L and 5 g/L;
one or more vitamins at a concentration of between 0.001 g/L and 1 g/L;
one or more trace elements at a concentration of between 0.0001 mg/L and 10 mg/L;
one or more growth-stimulating and/or cell-signaling factors at a concentration of between 0 ng/ml and 100,000 ng/mL; and
one or more transport proteins at a concentration of between 0 g/L and 1 g/L.

63-65. (canceled)

66. The method of claim 34, wherein the plant protein composition is present at a concentration of about 200 mg/L to about 400 mg/L.

67-72. (canceled)

73. The method of claim 34, wherein the basal culture media is selected from the group comprising B8 media, Dulbecco's Modified Eagle Medium (DMEM), F12, and a mixture thereof.

74-81. (canceled)
